(19) United States
(12) Patent Application Publication
(10) Pub. No.: US 2024/0360142 A1
(43) Pub. Date: Oct. 31, 2024

(54) Multitargeting Agents For The Potential Treatment of Dementia With Lewy Bodies, Parkinson's Disease, and Alzheimer Disease With Lewy Bodies

(71) Applicant: Duquesne University of The Holy Spirit, Pittsburgh, PA (US)

(72) Inventors: Aleem Gangjee, Allison Park, PA (US); Rehana K. Leak, Pittsburgh, PA (US)

(21) Appl. No.: 18/644,844

(22) Filed: Apr. 24, 2024

Related U.S. Application Data
(60) Provisional application No. 63/498,117, filed on Apr. 25, 2023.

(51) Int. Cl. C07D 487/04; A61K 31/519; A61P 25/28

(52) U.S. Cl. CPC C07D 487/04; A61K 31/519; A61P 25/28

(57) ABSTRACT

A compound having the structure of AGN5 or AGN11

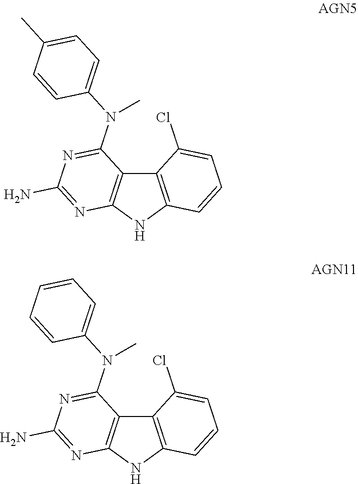 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

is provided. A composition comprising either AGN5 or AGN11 and a pharmaceutically acceptable carrier is provided. A method of treating a patient having dementia with Lewy Bodies, Parkinson's Disease, and Alzheimer Disease with Lewy Bodies comprising administering to said patient a therapeutically effective amount of the compound AGN5, AGN11, or the composition comprising AGN5 or AGN11 and a pharmaceutically acceptable carrier.

### CROSS-REFERENCE TO RELATED APPLICATION

This utility non-provisional patent application claims the benefit of priority to U.S. Provisional Patent Application Ser. No. 63/498,117, filed Apr. 25, 2023. The entire contents of U.S. Provisional Patent Application Ser. No. 63/498,117 are incorporated by reference into this utility non-provisional patent application as if fully rewritten herein.

### STATEMENT REGARDING FEDERALLY SPONSORED RESEARCH OR DEVELOPMENT

This invention was made with government support under Grant No. R21AG068608 awarded by the National Institutes of Health and CA142868 awarded by the National Cancer Institute. The government has certain rights in the invention.

### BACKGROUND OF THE INVENTION

### 1. Field of the Invention

This invention provides compounds AGN-5 and AGN-11, tricylic pyrimido[4,5-b]indole analogs, as described herein, for use as multitargeting agents for the treatment of dementia with Lewy bodies, Parkinson's disease, and Alzheimer disease with Lewy bodies. Compounds AGN5 and AGN11 are useful in treating diseases with Lewy Bodies.

### 2. Background Art

Lewy body disorders are a family of neurodegenerative diseases associated with debilitating problems in cognition, movement, and mood. Lewy body disorders include Parkinson's disease (PD) and dementia with Lewy bodies (DLB). PD is the most common movement disorder. However, DLB is the second most common cause of dementia, and nearly half of patients with Alzheimer's disease display coexisting Lewy and amyloid pathology in the brain. Thus, compounds that successfully reduce Lewy pathology are expected to exert a broad positive impact in the clinic. The major hallmark of these disorders, the Lewy body, is filled with abnormal, fibrillar forms of the protein alpha-synuclein. Fibrillar alpha-synuclein interferes with cellular function, including the transport of cargo along the tracks formed by the “cellular skeleton”—the microtubules.

### SUMMARY OF THE INVENTION

The present invention provides multitargeting compounds AGN 5 (“5”, “AG161-41”) and AGN11 (“11”, “AG161-47”), which decrease the extent of Lewy-like inclusion formation through nontoxic and relatively subtle interactions with the microtubule protein alpha-tubulin, as well as more potent interactions with kinase proteins involved in the development of Lewy inclusions (e.g., Abl-1, LRRK2, GSK3β). As used herein, compound AG161-41 is also identified as compound AGN5 or “5”, and compound AG161-47 is also identified as compound AGN11 or “11”.

In certain embodiments of this invention, a compound having the structure of either AGN5 or AGN11:

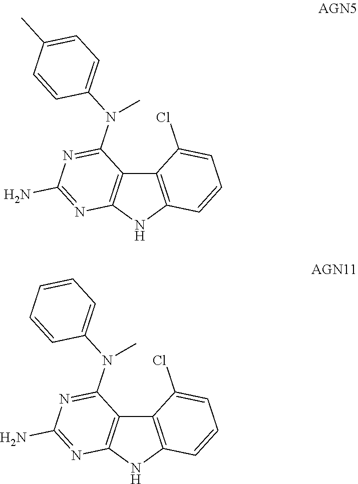 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

is provided.

Another embodiment of this invention provides a composition comprising a compound having the structure of either AGN5 or AGN11:

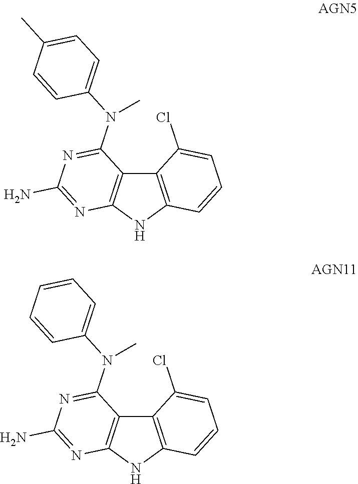 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

and a pharmaceutically acceptable carrier.

In yet another embodiment of this invention, a method is provided for treating a patient having a disease selected from the group consisting of dementia with Lewy Bodies, Parkinson's Disease, and Alzheimer Disease with Lewy Bodies comprising administering to said patient a therapeutically effective amount of a compound having a structure of either AGN5 or AGN11:

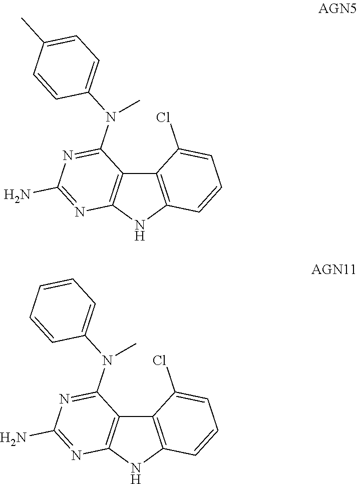 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

for treating said patient.

In another embodiment of this invention, a method is disclosed for treating a patient having a disease selected from the group consisting of dementia with Lewy Bodies, Parkinson's Disease, and Alzheimer Disease with Lewy Bodies comprising administering to said patient a therapeutically effective amount of a composition comprising a compound having a structure of either AGN5 or AGN11:

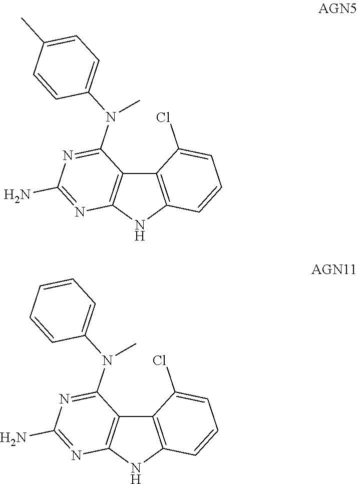 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

and a pharmaceutically acceptable carrier, for treating said patient.

In another embodiment of this invention, a method is disclosed for treating a patient affected with a Lewy body disease comprising administering to said patient a therapeutically effective amount of a compound having a structure of either AGN5 or AGN11:

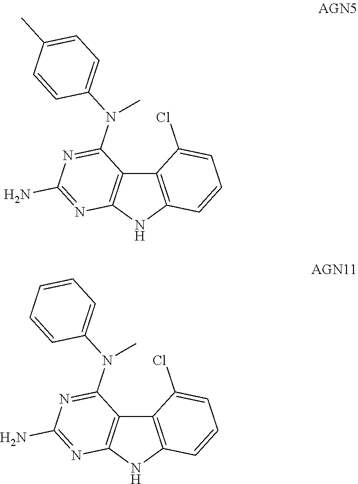 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

for treating said patient.

In another embodiment of this invention, a method is provided for treating a patient having a disease selected from the group consisting of dementia with Lewy Bodies, Parkinson's Disease, and Alzheimer Disease with Lewy Bodies comprising administering to said patient a therapeutically effective amount of a composition comprising a compound having a structure of either AGN5 or AGN11:

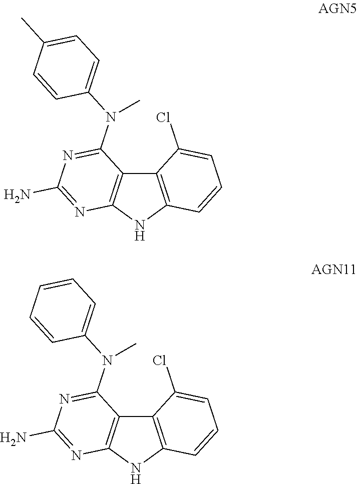 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

and a pharmaceutically acceptable carrier, for treating said patient.

In certain embodiments of this invention, a method is provided for treating a patient having a Lewy body disease comprising administering to said patient a therapeutically effective amount of a compound having a structure of either AGN5 or AGN11:

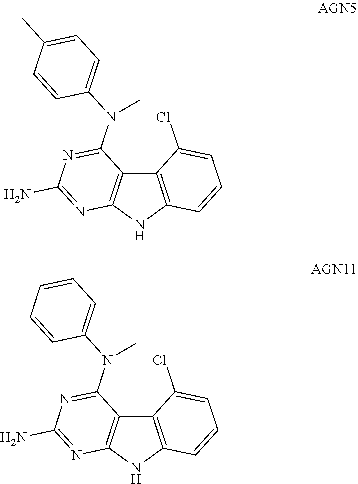 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

for treating said patient.

In another embodiment of this invention, a method is provided for treating a patient having a Lewy body disease comprising administering to said patient a therapeutically effective amount of a composition comprising a compound having a structure of either AGN5 or AGN11:

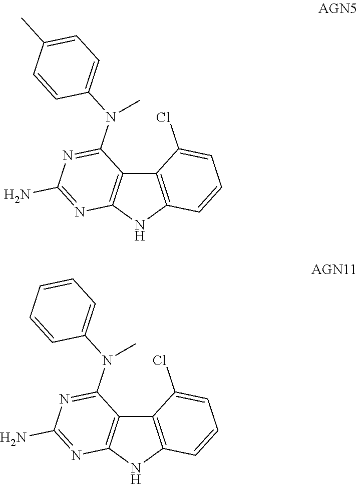 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

and a pharmaceutically acceptable carrier, for treating said patient.

In another embodiment of this invention, a compound having the structure of AG161-41 AG161-47, AG182-126, or AG185-174 is provided:

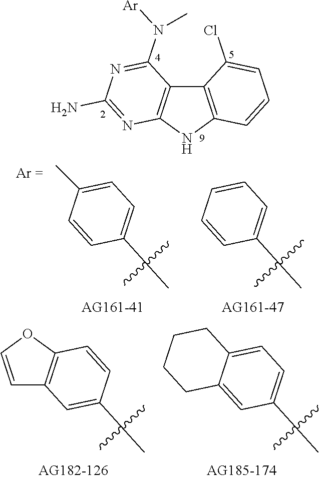

In another embodiment of this invention, a method is provided for treating a patient having a disease selected from the group consisting of dementia with Lewy Bodies, Parkinson's Disease, and Alzheimer Disease with Lewy Bodies comprising administering to said patient a therapeutically effective amount of a compound having a structure of either AG161-41, AG161-47, AG182-126, or AG185-174, for treating said patient.

### BRIEF DESCRIPTION OF THE DRAWINGS

FIG. 1A shows the impact of pyrimido[4,5-b]indole AG161-47 on α-synucleinopathy in vitro. Primary hippocampal cultures were treated with preformed α-synuclein fibrils and AG161-47 for 10 days in vitro before immunoblotting for hyperphosphorylated α-synuclein (pSer129) as a marker of Lewy-like pathology.

FIG. 1B shows levels of pSer129 α-synuclein as a fraction of pan α-synuclein.

FIG. 1C. shows pSer129 as a fraction of total protein concentrations.

FIG. 1D shows pSer129 as a fraction of total protein concentrations.

FIG. 1E shows total protein levels.

FIG. 1F shows exogenously applied preformed fibrils.

FIG. 1G shows endogenous α-synuclein (i.e., excluding the synthetic fibrils).

FIG. 2A shows the impact of pyrimido[4,5-b]indole AG161-47 on α-synucleinopathy in vitro. Primary hippocampal cultures were treated with preformed α-synuclein fibrils and AG161-47 for 10 days in vitro before immunostaining for hyperphosphorylated α-synuclein (pSer129) as a marker of Lewy-like pathology and the pan-neuronal marker Hoechst as a viability marker. Shown are viable Hoechst⁺ cell counts per field of view under a 20× objective.

FIG. 2B shows nonviable Hoechst⁺ fragmented/condensed nuclei per field of view under a 20× objective.

FIG. 2C shows Ser129⁺ object counts per Hoechst⁺ cell.

FIG. 2D shows the area occupied by pSer129⁺ objects (area fraction) per Hoechst⁺ cell.

FIG. 2E shows Log₁₀ pSer129⁺ area fraction per Hoechst⁺ cell.

FIG. 3A shows the impact of pyrimido[4,5-b]indole AG161-41 on α-synucleinopathy in vitro. Primary hippocampal cultures were treated with preformed α-synuclein fibrils and AG161-41 for 10 days in vitro before immunostaining for hyperphosphorylated α-synuclein (pSer129) as a marker of Lewy-like pathology. Shown are viable Hoechst⁺ cell counts per field of view under a 20× objective.

FIG. 3B shows the area occupied by pSer129⁺ objects (area fraction) per Hoechst⁺ cell. AG161-47 slightly lowers this measure of α-synucleinopathy.

FIG. 3C shows average sizes of individual pSer129⁺ objects.

FIG. 4A shows the lack of severe impact of pyrimido[4,5-b]indole AG161-47 on post-translational markers of microtubule stability.

FIG. 4B shows quantification of detyrosinated α-tubulin as a fraction of pan α-tubulin.

FIG. 4C shows tyrosinated α-tubulin as a fraction of pan α-tubulin.

FIG. 4D shows tyrosinated α-tubulin as a fraction of pan α-tubulin.

FIG. 4E shows acetylated α-tubulin as a fraction of pan α-tubulin.

FIG. 4F shows acetylated α-tubulin as a fraction of pan α-tubulin.

FIG. 4G shows acetylated α-tubulin as a fraction of pan α-tubulin.

FIG. 5A shows that AG161-47 uncouples the link between post-translational markers of microtubule stability status and Lewy-related pathologies. Shown is the inverse correlation between tyrosinated α-tubulin (marker of nonstable microtubules) and acetylated α-tubulin (marker of stable microtubules), in the presence of preformed α-synuclein fibrils.

FIG. 5B shows an inverse correlation between tyrosinated α-tubulin (marker of nonstable microtubules) and acetylated α-tubulin (marker of stable microtubules) in the presence of nocodazole.

FIG. 5C shows an inverse correlation between tyrosinated α-tubulin (marker of nonstable microtubules) and acetylated α-tubulin (marker of stable microtubules) in the presence of AG161-47.

FIG. 5D shows an unexpected positive correlation between pSer129 α-synuclein and the fraction of α-tubulin that was detyrosinated (marker of stable microtubules), in the presence of preformed α-synuclein fibrils.

FIG. 5E shows that AG161-47 blocked the association between pSer129 levels and the fraction of α-tubulin that was detyrosinated (marker of stable microtubules).

FIG. 5F shows that AG161-47 blocked the association between pSer129 α-synuclein and the total mass of acetylated (stable) α-tubulin.

FIG. 6A shows that AG161-47 does not lower weekly bodyweights of mice, shown in grams and as % change in bodyweight over time, in an in vivo model of limbic-centered Lewy body disease.

FIG. 6B shows that AG161-47 lowers locomotor activity in an open in an in vivo model of limbic-centered Lewy body disease. After six weeks of daily tablet consumption, a battery of behavior tests was completed, including the open field test. Shown here are each animal's mean speed, number of rears, and distance traveled in the open field.

FIG. 7A shows that 5 mg/kg AG161-47 slightly lowers insoluble pathological α-synuclein in an in vivo model of limbic-centered Lewy body disease. Panels from left to right show quantification of the Total Protein REVERT stain as the loading control, insoluble pSer129 α-synuclein as a fraction of total protein, insoluble pan α-synuclein as a fraction of total protein, and insoluble pSer129 α-synuclein as a fraction of pan α-synuclein.

FIG. 7B shows correlations between insoluble pan α-synuclein levels and the fraction of α-tubulin that was tyrosinated or the levels of acetylated α-tubulin in fibril-infused mice.

FIG. 7C shows the positive correlation of insoluble pSer129 α-synuclein levels with the fraction of α-tubulin that was acetylated (marker of microtubule stability) in fibril-infused animals. AG161-47 uncouples this link at a dose of 5 mg/kg.

FIG. 7D shows a near-significant correlation of detyrosinated α-tubulin levels with the fraction of insoluble α-synuclein that was hyperphosphorylated in fibril-infused mice. AG161-47 uncouples this link at a dose of 5 mg/kg.

FIG. 7E shows the inverse correlation of mean locomotor speed with insoluble pan α-synuclein in fibril-infused animals. AG161-47 uncouples this link at a dose of 5 mg/kg.

FIG. 8 shows a synthesis route for compounds AG182-126 and AG185-174 with the following reaction conditions: AG182-126: (a) i) N-methylbenzofuran-5-amine 2, n-BuOH, conc. HCl, 150° C., 4 hours, microwave reactor ii) 1N NaOH, reflux, 4 hours, 25%; AG185-174: (b) i) N-methyl-5,6,7,8-tetrahydronaphthalen-2-amine 3, i-PrOH, conc. HCl, reflux, 120 hours ii) 1N NaOH, reflux, 4 hours, 30%.

FIG. 9A shows the chemical structures of the indicated compounds used to evaluate the impact of microtubule stabilization or destabilization on Lewy-like pathologies in vitro.

FIG. 9B shows the impact of pyrimido[4,5-b]indoles on α-synucleinopathy in vitro. Primary hippocampal cultures were treated with preformed α-synuclein fibrils and the compounds indicated for 10 days in vitro before immunostaining for hyperphosphorylated α-synuclein (pSer129) as a marker of Lewy-like pathology, and the pan-neuronal marker βIII-tubulin as a viability marker.

FIG. 9C shows the impact of pyrimido[4,5-b]indoles on α-synucleinopathy in vitro.

FIG. 9D shows the impact of pyrimido[4,5-b]indoles on the pan-neuronal marker βIII-tubulin as a viability marker.

FIG. 9E shows the impact of pyrimido[4,5-b]indoles on the pan-neuronal marker βIII-tubulin as a viability marker.

FIG. 9F shows the impact of pyrimido[4,5-b]indoles on α-synucleinopathy in vitro.

FIG. 9G shows the impact of pyrimido[4,5-b]indoles on α-synucleinopathy in vitro.

FIG. 10A shows the impact of AG161-47 on acetylated α-tubulin in vivo in a model of limbic-centered Lewy body disease. Levels of acetylated α-tubulin as a fraction of pan α-tubulin and total protein are shown.

FIG. 10B shows the impact of AG161-47 on detyrosinated α-tubulin in vivo in a model of limbic-centered Lewy body disease. Levels of detyrosinated α-tubulin as a fraction of pan α-tubulin and total proteins are shown.

FIG. 10C shows the impact of AG161-47 on tyrosinated α-tubulin in vivo in a model of limbic-centered Lewy body disease. Levels of tyrosinated α-tubulin as a fraction of pan α-tubulin and total protein are shown.

FIG. 11A shows the impact of biological sex on tyrosinated α-tubulin in the human amygdala. Amygdala samples harvested postmortem from individuals diagnosed with or without Lewy body disorders were subjected to SDS-PAGE and immunoblotting for tyrosinated α-tubulin.

FIG. 11B shows the impact of biological sex on total protein levels in the human amygdala. Amygdala samples harvested postmortem from individuals diagnosed with or without Lewy body disorders were subjected to SDS-PAGE and immunoblotting for total protein levels.

FIG. 11C shows the impact of biological sex on pan α-tubulin in the human amygdala. Amygdala samples harvested postmortem from individuals diagnosed with or without Lewy body disorders were subjected to SDS-PAGE and immunoblotting for pan α-tubulin.

FIG. 11D shows the impact of biological sex on the fraction of α-tubulin that is tyrosinated in the human amygdala. Amygdala samples harvested postmortem from individuals diagnosed with or without Lewy body disorders were subjected to SDS-PAGE and immunoblotting for tyrosinated α-tubulin.

### DETAILED DESCRIPTION OF THE INVENTION

The present invention provides multitargeting compounds AGN 5 (“5”, “AG161-41”) and AGN11 (“11”, “AG161-47”), which decrease the extent of Lewy inclusion formation through nontoxic and relatively subtle interactions with the microtubule protein alpha-tubulin, as well as more potent interactions with kinase proteins involved in the development of Lewy inclusions (e.g., Abl-1, LRRK2, GSK3β).

As used herein, the term “patient” refers to any member of the animal kingdom, including but not limited to, Homo sapiens.

As used herein, the term “pharmaceutically acceptable carrier” refers to any acceptable pharmaceutical carrier that is known by those persons of ordinary skill in the art, and incudes, for example but not limited to, dextrose or saline.

As used herein, the term “therapeutically effective amount” refers to that amount of a compound or composition that is required to bring about a desired effect or treatment of a patient.

In certain embodiments of this invention, a compound having the structure of either AGN5 or AGN11:

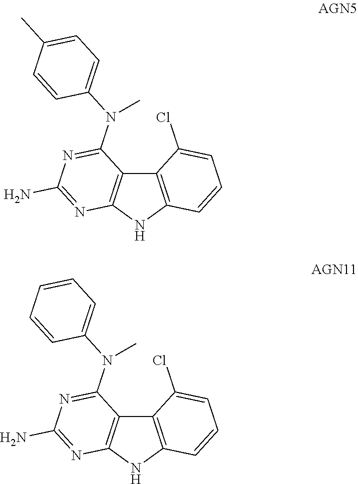 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

is provided.

Another embodiment of this invention provides a composition comprising a compound having the structure of either AGN5 or AGN11:

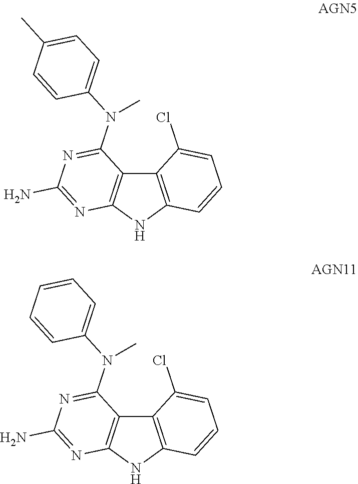 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

and a pharmaceutically acceptable carrier.

In yet another embodiment of this invention, a method is provided for treating a patient having a disease selected from the group consisting of dementia with Lewy Bodies, Parkinson's Disease, and Alzheimer Disease with Lewy Bodies comprising administering to said patient a therapeutically effective amount of a compound having a structure of either AGN5 or AGN11:

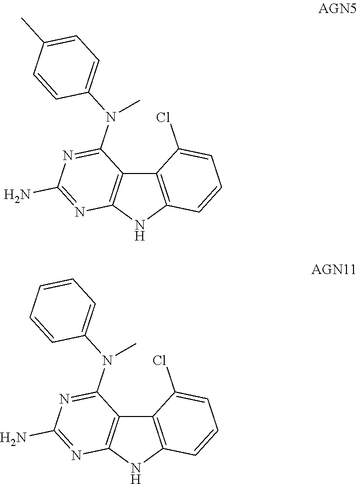 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

for treating said patient.

In another embodiment of this invention, a method is disclosed for treating a patient having a disease selected from the group consisting of dementia with Lewy Bodies, Parkinson's Disease, and Alzheimer Disease with Lewy Bodies comprising administering to said patient a therapeutically effective amount of a composition comprising a compound having a structure of either AGN5 or AGN11:

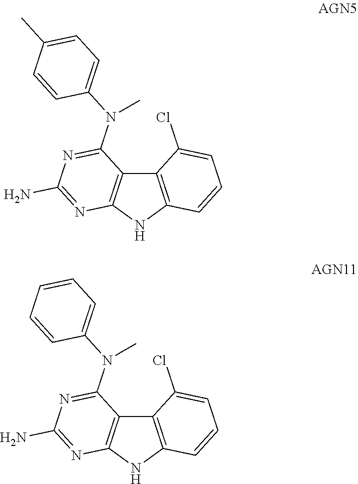 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

and a pharmaceutically acceptable carrier, for treating said patient.

In another embodiment of this invention, a method is disclosed for treating a patient affected with a Lewy body disease comprising administering to said patient a therapeutically effective amount of a compound having a structure of either AGN5 or AGN11:

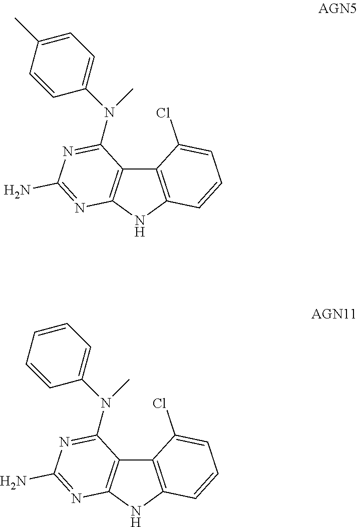 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

for treating said patient.

In another embodiment of this invention, a method is provided for treating a patient having a disease selected from the group consisting of dementia with Lewy Bodies, Parkinson's Disease, and Alzheimer Disease with Lewy Bodies comprising administering to said patient a therapeutically effective amount of a composition comprising a compound having a structure of either AGN5 or AGN11:

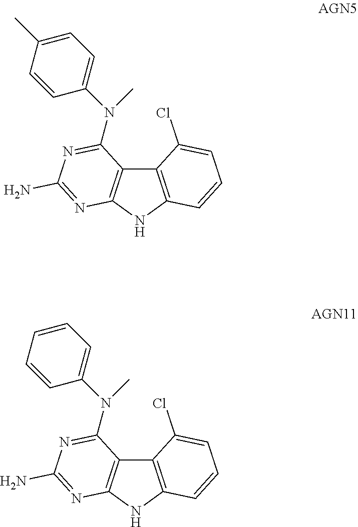 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

and a pharmaceutically acceptable carrier, for treating said patient.

In certain embodiments of this invention, a method is provided for treating a patient having a Lewy body disease comprising administering to said patient a therapeutically effective amount of a compound having a structure of either AGN5 or AGN11:

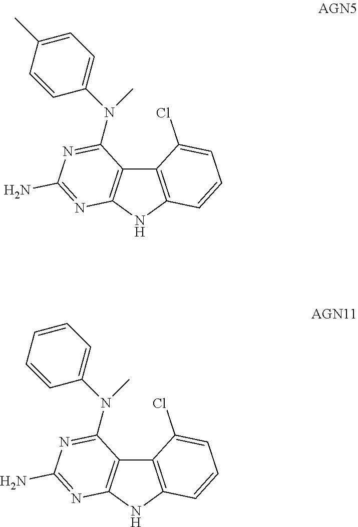 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

for treating said patient.

In another embodiment of this invention, a method is provided for treating a patient having a Lewy body disease comprising administering to said patient a therapeutically effective amount of a composition comprising a compound having a structure of either AGN5 or AGN11:

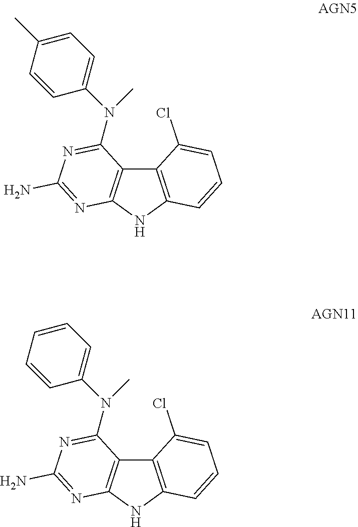 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

and a pharmaceutically acceptable carrier, for treating said patient.

In another embodiment of this invention, a compound having the structure of AG161-41 AG161-47, AG182-126, or AG185-174 is provided:

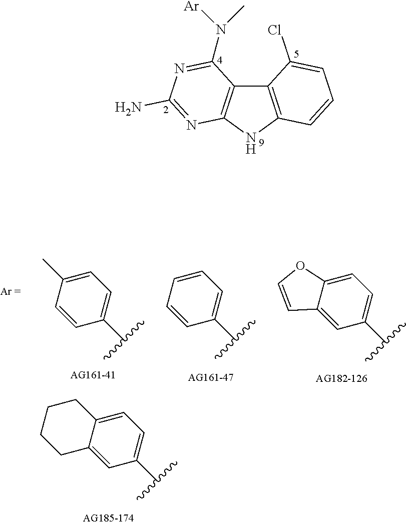

In another embodiment of this invention, a method is provided for treating a patient having a disease selected from the group consisting of dementia with Lewy Bodies, Parkinson's Disease, and Alzheimer Disease with Lewy Bodies comprising administering to said patient a therapeutically effective amount of a compound having a structure of either AG161-41, AG161-47, AG182-126, or AG185-174, for treating said patient.

As used herein, compound AG161-41 is also identified as compound AGN5 or “5”, and compound AG161-47 is also identified as compound AGN11 or “11”.

In Lewy body disorders, α-synuclein aggregates are thought to destabilize the microtubular framework. However, microtubules are a multifaceted therapeutic target, as rigidity afforded by potent microtubule stabilizers may be toxic. Therefore, we report dual-acting, mild microtubule-stabilizing/destabilizing agents in experimental Lewy body disease. Among the candidates, tricylic pyrimido[4,5-b]indole analogs AG161-47 and AG161-41 (4 μM) reduced preformed fibril-seeded α-synucleinopathy without toxicity in primary rat hippocampal cultures. The ability of AG161-47 to lower α-synucleinopathy and nuclear fragmentation was interrupted by sublethal (5 nM) concentrations of the microtubule disruptor nocodazole. AG161-47 was then administered in a versatile, new tablet formulation to male mice infused with 5 μg fibrils in the bulbar anterior olfactory nucleus, to model limbic Lewy body disease. Mice voluntarily consumed tablets with AG161-47—without need for daily oral gavage or invasive injections. The fraction of insoluble α-synuclein hyperphosphorylated at serine 129 was modestly lowered by 5 mg/kg AG161-47.

Lewy body disorders are characterized by aggregation of the abundant protein α-synuclein in hallmark neuronal inclusions. There are numerous functions attributed to α-synuclein, ranging from neurotransmission and cell survival to axonal transport (Anderson et al., 2019; Chu et al., 2012; Freundt et al., 2012; Toba et al., 2017; Volpicelli-Daley, 2017)). See reference section herein for complete titles and publication data for all referenced works. For example, α-synuclein is present in axon terminals, where it supports vesicle trafficking (Cooper et al., 2006; Gitler et al., 2008; Scott & Roy, 2012). Evidence also favors a functional association between α-synuclein and microtubules, the ‘conveyer belts’ of the cytoskeleton (Baas et al., 2016; Brunden et al., 2017; Lee et al., 2006; Toba et al., 2017). Thus, destabilization of microtubules has been argued to contribute to disease progression in Lewy body disorders (Bendiske et al., 2002; Cartelli & Cappelletti, 2017; Cartelli et al., 2013; Cartelli et al., 2012; Choi et al., 2011; Qureshi & Paudel, 2011; Ren et al., 2005).

Microtubules are assemblies of α-tubulin and β-tubulin heteropolymers, ranging in structure from rigid to flexible or relaxed (Baas & Black, 1990; Brouhard & Rice, 2018; Etienne-Manneville, 2010; Gudimchuk & McIntosh, 2021; Janke & Magiera, 2020; Manka & Moores, 2018; Mitchison & Kirschner, 1984). Microtubules can quickly alternate between growing and shrinking phases—allowing reorganization of the cytoskeleton where needed (Brouhard & Rice, 2018; Dogterom & Koenderink, 2019; Gudimchuk & McIntosh, 2021; Janke & Magiera, 2020; Manka & Moores, 2018; Parato & Bartolini, 2021). This spatiotemporally orchestrated process is known as dynamic instability and, if not coordinated, can culminate in ‘catastrophe’ events with unraveling of the microtubular scaffold by rapid depolymerization of entire lengths of nonstabilized and moderately stabilized microtubules (Baas & Black, 1990; Baas et al., 2016; Cartelli et al., 2016; Chauvin & Sobel, 2015; Niwa, 2015; Pellegrini et al., 2017; Wu et al., 2020). Microtubule dynamicity endows the cell with the ability to quickly synthesize and remove its microtubule tracks for passing cargo, to retract and extend processes, or to adjust the tensile strength of somata and processes (Canty et al., 2021; Hirokawa et al., 2009; Lu & Gelfand, 2017). Cycles of polymerization and depolymerization are particularly essential for neuronal viability and must be carefully titrated to avoid interruption of axonal transport. For partly these reasons, modulation of microtubule stability dynamics in the central nervous system is a double-edged sword, and excessive modification of microtubular structure—including stabilization—will have to be avoided in neurodegenerative therapeutic strategies.

The dynamicity of microtubular structures is regulated by a battery of post-translational modifications and interactions with microtubule-associated proteins and cargo transporters (Brouhard & Rice, 2018; Dogterom & Koenderink, 2019; Gudimchuk & McIntosh, 2021; Janke & Magiera, 2020; Manka & Moores, 2018; Naren et al., 2023; Pero et al., 2023). Native α-synuclein may also directly and indirectly interact with microtubules, serving as a microtubule ‘dynamase’ that regulates nucleation as well as catastrophe events (Carnwath et al., 2018; Cartelli et al., 2016; Oikawa et al., 2016; Prots et al., 2013; Volpicelli-Daley, 2017; Volpicelli-Daley, Gamble, et al., 2014). However, proteinaceous aggregates interfere with transport of cargo and destabilize microtubules, and α-synuclein aggregation may therefore disrupt axonal transport and induce microtubule depolymerization (Baas et al., 2016; Carnwath et al., 2018; Cartelli et al., 2016; Lee et al., 2006; Mazzetti et al., 2023; Oikawa et al., 2016; Prots et al., 2013; Volpicelli-Daley, 2017; Volpicelli-Daley, Gamble, et al., 2014).

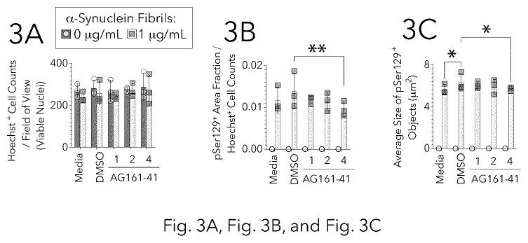

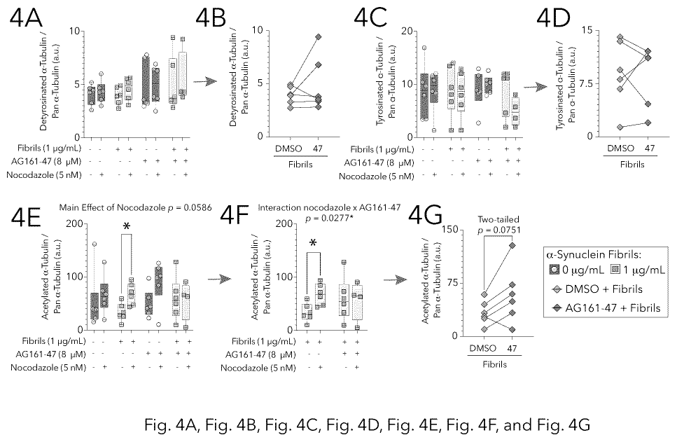

Four pyrimido[4,5-b]indole tricyclic compounds of this invention designed to exert subtle microtubule modulating effects were evaluated in this invention. We demonstrate that Compound 3, defined here as AG161-47, accelerated and amplified the net in vitro assembly of tubulin purified from porcine brain (albeit not to the same degree as paclitaxel) and elicited both stabilization and relaxation of microtubules in HeLa cells (FIGS. 3A, and 4D). Here, we report that AG161-47 lowered α-synucleinopathy and nuclear condensation and fragmentation after preformed fibril exposure in primary hippocampal cultures and lowered levels of insoluble, hyperphosphorylated α-synuclein in male mice infused with preformed fibrils in the olfactory bulb/anterior olfactory nucleus (OB/AON). For the animal studies, we developed and tested a new chewable tablet formulation as a means of noninvasive delivery of orally bioavailable drugs. Neither fibril nor AG161-47 exposure induced robust changes in post-translational markers of microtubule stability in vivo or in vitro. However, markers of microtubule stability status were correlated with α-synucleinopathy in fibril-treated mice and primary hippocampal cultures. In both the in vivo and in vitro model, some of these relationships were uncoupled by exposure to AG161-47. In postmortem samples of the human OB, we also did not observe that Lewy body disease affected markers of microtubule stability status. Rather, biological sex exerted a stronger influence on the latter measures.

### Materials and Methods

### Experimental Syntheses

Chemicals were purchased at the highest purity available from ThermoFisher Scientific or Sigma-Aldrich. All evaporations were carried out in vacuum with a rotary evaporator. Analytical samples were dried in vacuo in a CHEM-DRY drying apparatus over P₂O₅ at 50° C. Melting points were determined either using a MPA100 OptiMelt automated melting point system and are uncorrected. Nuclear magnetic resonance spectra for proton (¹H NMR) were recorded on the Bruker Avance II 400 (400 MHz) or Bruker Advance II 500 (500 MHz) NMR systems with TopSpin processing software. ¹H spectra were referenced to tetramethylsilane and trifluoroacetic acid, respectively, as the internal standards to express the chemical shift values (δ) in ppm (parts per million): s, singlet; d, doublet; dd, doublet of doublet; t, triplet; q, quartet; m, multiplet; br, broad singlet; td, triplet of doublet; dt, doublet of triplet; quin, quintet. ¹H NMR of alkyl CH₂s of target compounds merged with dimethyl sulfoxide (DMSO) and water peaks have been omitted from the experiment whenever not visible in ¹H NMR.

Thin-layer chromatography (TLC) was performed on Whatman® PE SIL G/UV254 flexible silica gel plates and the spots were visualized under 254 and 365 nm ultraviolet illumination. Proportions of solvents used for TLC are by volume. All analytical samples were homogeneous on TLC in at least two different solvent systems. Column chromatography was performed on the silica gel (70 to 230 meshes, ThermoFisher Scientific) column. Flash chromatography was carried out on the CombiFlash® Rf systems, model COMBIFLASH RF. Pre-packed RediSep® Rf normal-phase flash columns (230 to 400 meshes) of various sizes were used. The amount (weight) of silica gel/celite for column chromatography was in the range of 5-10 times the amount (weight) of the crude reaction mixture being separated. Mass spectrometry was performed on Advion Expression® compact mass spectrometers (CMS). High Performance Liquid Chromatography (HPLC) was performed on Vanquish Core HPLC System (ThermoFisher Scientific) components: Multiwavelength detector CG (model code: VC-D12-α-01), Split sampler C (model code: VC-A13-A-02), Quaternary pump C (model code: VC-P20-A-01), Column compartment C (model code: VC-C10-A-03), Fraction Collector F (model code: VF-F11-A-01). The mobile phase was an aqueous blend of 0.1% formic acid in water with a miscible, polar organic solvent acetonitrile.

N-(4,5-dichloro-9H-pyrimido[4,5-b]indol-2-yl)pivalamide (Compound 1) was synthesized according to the published procedure (Gangjee et al., 2010; Rohena et al., 2016)

### N-methylbenzofuran-5-amine (2)

Benzofuran-5-amine (500 mg, 3.76 mmol; CC07114DA, ThermoFisher Scientific) was added to a solution of NaOMe (1.1 g, 18.78 mmol) in 10 mL MeOH. The resulting solution was poured into a suspension of paraformaldehyde (157.85 mg, 5.26 mmol) in MeOH (10 mL). The reaction mixture was stirred overnight at room temperature. Sodium borohydride (142 mg, 3.76 mmol) was then added, and the reaction mixture was heated at reflux for 2 hours. After cooling, silica gel was added to the reaction mixture and the solvent was removed under reduced pressure to provide a plug. The plug was transferred on top of a column packed with silica gel and was eluted with hexane:ethyl acetate as the eluent. Fractions containing the product (TLC) were pooled and evaporated to afford 356 mg (64%) of Compound 2 as a brown liquid. TLC Rf=0.32 (EtOAc:hexane 1:4 and 2 drops NH₄OH); ¹H NMR (500 MHz, DMSO-d₆) δ 7.79 (d, J=2.1 Hz, 1H), 7.29 (d, J=8.7 Hz, 1H), 6.75 (dd, J=2.1, 1.0 Hz, 1H), 6.65-6.58 (m, 2H), 5.46 (q, J=5.2 Hz, 1H, exch), 2.68 (d, J=5.2 Hz, 3H).

### N-methyl-5,6,7,8-tetrahydronaphthalen-2-amine (3)

5,6,7,8-Tetrahydronaphthalen-2-amine (2 g, 13.58 mmol; A453457, Ambeed) was added to a solution of NaOMe (3.67 g, 67.9 mmol) in 20 mL of MeOH. The resulting solution was poured into a suspension of paraformaldehyde (571 mg, 19 mmol) in MeOH (20 mL). The reaction mixture was stirred overnight at room temperature. Sodium borohydride was then added, and the reaction mixture was heated at reflux for 2 hours. After completion of the reaction, 1M KOH was added to the reaction mixture and the product was extracted using EtOAc. The combined organic layers were dried over sodium sulfate and silica gel was added. Next, the solvent was removed under reduced pressure to provide a plug. The plug was transferred on top of a column packed with silica gel and was eluted with hexane:ethyl acetate. Fractions containing the product (TLC) were pooled and evaporated to afford 875 mg (40%) of 3 as an orange liquid. TLC Rf=0.6 (EtOAc:hexane, 1:2); ¹H NMR (400 MHz, DMSO-d₆) δ 6.75 (d, J=8.1 Hz, 1H), 6.32 (dd, J=8.1, 2.5 Hz, 1H), 6.21 (d, J=2.5 Hz, 1H), 5.26 (s, 1H), 2.65-2.53 (m, 7H), 1.68 (dq, J=6.7, 2.8 Hz, 4H).

### N⁴-(benzofuran-5-yl)-5-chloro-N⁴-methyl-9H-pyrimido[4,5-b]indole-2,4-diamine (AG182-126)

N-methylbenzofuran-5-amine 2 (148 mg, 1.01 mmol) and 2 drops of concentrated HCl were added to a solution of 1 (113 mg, 0.33 mmol) in 15 mL of n-butanol. The reaction mixture was heated at 150° C. for 4 hours in a microwave reactor. After cooling to room temperature, 2 mL of 1N sodium hydroxide were added. The reaction was then heated at reflux for 4 hours. After cooling, silica gel was added to the reaction mixture and the solvent was removed under reduced pressure to provide a plug. The plug was transferred on top of a column packed with silica gel and was eluted with hexane:ethyl acetate as the eluent. Fractions containing the product (TLC) were pooled and evaporated to afford 31 mg (25%) of AG182-126 as a buff-colored powder. TLC Rf=0.54 (MeOH:DCM, 1:5 and 2 drops NH₄OH); mp 199-201° C.; ¹H NMR (500 MHz, DMSO-d₆) δ 11.77 (s, 1H), 7.88 (d, J=2.1 Hz, 1H), 7.38 (d, J=8.9 Hz, 1H), 7.27 (dd, J=8.0, 1.0 Hz, 1H), 7.17 (t, J=7.9 Hz, 1H), 7.10 (d, J=2.4 Hz, 1H), 7.02 (dd, J=7.8, 1.0 Hz, 1H), 6.89 (dd, J=9.0, 2.4 Hz, 1H), 6.82 (dd, J=2.2, 1.0 Hz, 1H), 6.53 (s, 2H), 3.41 (s, 3H). MS (ESI) calculated for C₁₉H₁₅ClN₅O [M+H]⁺, 364.09. Found: 364.4. HPLC purity>95% (290 nm).

### 5-chloro-N⁴-methyl-N⁴-(5,6,7,8-tetrahydronaphthalen-2-yl)-9H-pyrimido[4,5-b]indole-2,4-diamine (AG185-174)

N-methyl-5,6,7,8-tetrahydronaphthalen-2-amine 3 (358 mg, 2.22 mmol) and 2 drops of concentrated HCl were added to a solution of 1 (250 mg, 0.74 mmol) in 50 mL of i-propanol. The reaction mixture was heated at reflux for 120 hours and cooled to room temperature. The solvent was then evaporated to obtain a dark brown colored solid which was dissolved in i-propanol (10 mL), and 5 mL of 1N sodium hydroxide solution was added. The reaction was then heated at reflux for 4 hours. Silica gel was added to the reaction mixture, at four times the weight of the reaction mixture, and the solvent was removed under reduced pressure to provide a plug. The plug was transferred on top of a column packed with silica gel (twenty times the weight of plug) and was eluted with hexane:ethyl acetate as the eluent. Fractions containing the product (TLC) were pooled and evaporated to afford 84 mg (30%) of AG185-174 as a light brown powder. TLC Rf=0.15 (EtOAc:hexane, 1:2); mp 192-194° C.; ¹H NMR (400 MHz, DMSO-d₆) δ 11.77 (s, 1H), 7.28 (d, J=7.9 Hz, 1H), 7.19 (t, J=7.9 Hz, 1H), 7.05 (d, J=7.8 Hz, 1H), 6.81 (d, J=8.2 Hz, 1H), 6.56 (t, J=8.9 Hz, 4H), 3.26 (s, 3H), 2.60 (s, 4H), 1.67 (s, 4H). MS (ESI) calculated for C₂₁H₂₀ClN₅ [M+H]⁺, 378.14. Found: 378.5. HPLC purity 97.29% (350 nm).

### Molecular Modeling Protocol

Ligands to be docked were prepared using the LigPrep module in Maestro (Schrödinger) Schrödinger Release 2023-11) (Schrödinger Release 2023-1) (Schrödinger Release 2023-1) (Schrödinger Release 2023-1). The proteins of interest were prepared using the protein preparation workflow in Maestro. Next, the compounds were docked in the proteins, using the “induced fit” docking module in Maestro. Default settings were used for all three actions. The docked structures of the compounds were examined for relevant, top-scoring docked poses. The selected docked structures were then superimposed using the C-alpha atoms of the protein with Root Mean Square Deviation (RMSD) values<0.07.

### Preparation of Preformed α-Synuclein Fibrils

Preformed α-synuclein fibrils (PFFs) were synthesized and validated as described before, as a kind gift from Dr. Kelvin Luk's lab at the University of Pennsylvania (Polinski et al., 2018; Volpicelli-Daley, Luk, et al., 2014). This batch of PFFs has been used and described in four recent publications from the Leak lab (Bhatia et al., 2021; Bhatia et al., 2023; Miner et al., 2023; Miner et al., 2022). Before shipment to Duquesne University of The Holy Spirit, the fibrils were evaluated by sedimentation for high MW species, Thioflavin assays for ß-sheets, bicinchoninic protein concentration tests, and endotoxins tests (<0.11 pg lipopolysaccharides/μg protein, per a communication from Dr. Luk), according to established guidelines (Polinski et al., 2018; Volpicelli-Daley, Luk, et al., 2014). On the day of in vitro use, the fibrils were diluted from the stock concentration of 5 μg/L to 1 μg/L in neuronal media (ingredients listed below). PFFs were sonicated in a closed tube for 1 hour at 40 kHz in a water bath at room temperature, as described (Mason et al., 2016; Mason et al., 2019; Nouraei et al., 2018) (Bransonic series model M1800, Branson Ultrasonics Corporation).

### Primary Hippocampal Cultures

With Institutional Animal Care and Use Committee (IACUC) approval, primary hippocampal cultures were generated from postnatal day 0-2 rat pups, as described in our prior reports (Bhatia et al., 2021; Posimo et al., 2014). Briefly, brains were harvested from Sprague Dawley rat pups and immersed in Hibernate A (HA; BrainBits, LLC.). The hippocampi were dissected and treated with papain (20 U/mL; LS 3126, Worthington Biochemical Corporation) for 30 minutes at 37° C. and 5% CO₂ to reduce intercellular adhesion. Papain was neutralized with 0.5 U of DNAse solution (DN25, Sigma-Aldrich), and tissues were gently triturated with a 5 mL serological pipette to generate a homogeneous cell suspension. The cell suspensions were filtered through 40 μm cell strainers and then plated in 12-well (353043, Falcon) or 96-well plates (3595, Corning Costar) or 3.5 mm dishes (353001, Falcon Corning), coated with 66.7 μg/mL poly-D-lysine (P0899, Sigma-Aldrich) and 6.7 μg/mL mouse laminin (354232, Corning).

Neurons were plated in Neurobasal-A medium (10888022, ThermoFisher Scientific) with GlutaMAX (35-050-061, ThermoFisher Scientific), penicillin/streptomycin (15-140-122, ThermoFisher Scientific), B27 (17-504-044, ThermoFisher Scientific), and 10% fetal bovine serum (511550H, Atlanta Biologicals). Plating media were exchanged for neuronal media (same ingredients but without fetal bovine sera) at 18 hours. Cultures were incubated at 37° C. in 5% CO₂ and treated on day-in-vitro (DIV) 2 with the appropriate vehicle or test compounds, fibrils, and nocodazole (M1404, Sigma-Aldrich) in a 50% media exchange. For long-term cultures, 50% of the media was again refreshed 7 days post-treatment on DIV 9. Treated cell cultures were fixed or lysates were collected on DIV 12, as described below, unless indicated otherwise. For long-term survival neuronal cultures, the cells were exposed to fibrils and/or treatments from DIV 2 to DIV 12.

### Immunocytochemistry

On DIV 12, cells were fixed and permeabilized with a cocktail of 3% paraformaldehyde (Formal-Fixx, 9990244, ThermoFisher Scientific) and 2% sucrose in 0.1 M phosphate buffer, with or without the nonionic detergent Triton X-100 at 1% v/v. Permeabilized cells were blocked using LI-COR Intercept Blocking Buffer PBS (927-70001, LI-COR) and immunostained as described previously (Bhatia et al., 2021; Posimo et al., 2014). Plates were imaged and quantified as In-Cell Westerns using an Odyssey imager (LI-COR) and LI-COR ImageStudio software (Ver. 5.2.5), or imaged at higher resolution on an epifluorescence microscope (Olympus IX73, B&B Microscopes). Wells subjected to all staining procedures but with omission of the primary antibody were used for background subtraction calculations in the In-Cell Western analyses.

Automated image analysis on blind-coded images was performed with Olympus cellSens software (Version 2.3). All nuclei were labeled with the pan-nuclear marker Hoechst 33258 (bisBenzimide, 5 μg/mL; B1155, Sigma-Aldrich). For viable cell counts, clusters of Hoechst⁺ nuclei with an area>500 μm², nuclei with an area<20 μm², or nuclei with a mean grey intensity (MGI)>600 arbitrary units (a.u.) in cellSens were excluded a priori as evidence of cell death and chromatin condensation (Eidet et al., 2014). Nuclei with areas<20 μm² and MGI>600 α.u. were thus defined as nonviable cells. For quantification of area fractions, cellSens divides the total number of pixels with target staining by the total number of pixels in the field of view and multiplies the result by 100 to generate a percentage value. The average area of the target object was divided by viable cell counts to obtain average inclusion size per cell. Thresholds were kept uniform across individual experiments. Primary antibodies are listed in Table 1. Highly cross-adsorbed secondary antibodies were used at concentrations of 1.07 μg/mL (Jackson Laboratories) and are listed in Table 2.

For cold methanol fixation, primary hippocampal cultures plated on glass slides were exposed to ice-cold methanol (−20° C.) for 15 minutes on DIV 4. Cells were then permeabilized and blocked using LI-COR Intercept Blocking Buffer PBS (927-70001, LI-COR) with 0.3% Triton X-100 and immunostained as described above. After immunostaining, coverslips were applied using FluoroMount-G (0100-01, SouthernBiotech).

### SDS-PAGE and Immunoblotting

For in vitro studies, 1× Cell Lysis Buffer was prepared (9803, Cell Signaling Technologies) supplemented with 1% protease inhibitor cocktail (P8340, Sigma-Aldrich), 1 mM phenylmethylsulfonylfluoride (PMSF; P7626, Sigma-Aldrich), 1% protease inhibitor cocktail (In-house, 2 mM imidazole, 1 mM sodium fluoride, and 1 mM sodium orthovanadate), and 10 mM sodium fluoride (S7920, Sigma-Aldrich). Media were removed on DIV 12 and ice-cold cell lysis buffer was added to the cell culture wells. Plates were then incubated for 10 minutes on wet ice, and cells were scraped and sonicated (XL2020, Misonix Inc.). Protein content was determined by the bicinchoninic acid protein assay (23225, ThermoFisher Scientific) or NanoDrop One^{C} (NanoDrop One C, ThermoFisher Scientific). Sample loading buffer (928-40004, LI-COR) containing 10% fresh β-mercaptoethanol was added at a 1:3 dilution to each sample.

The same protocol for cell lysis and protein extraction was used for mouse and human samples prior to immunoblotting. Ten micrograms of protein were loaded onto SDS-polyacrylamide gels and transferred to polyvinylidene difluoride (PVDF; Immobilon-FL PVDF, IPFL0010, Millipore) or nitrocellulose membranes (926-31092, LI-COR). Membranes were dried to minimize protein loss and PVDF membranes were rehydrated in methanol, whereas nitrocellulose membranes were rehydrated in water. When probing α-synuclein, 12% acrylamide gels were subjected to SDS-PAGE for >3 hours to improve spatial resolution. For all other proteins, 10% acrylamide gels were used. After transfer, the dried and rehydrated membranes were also fixed in 4% formalin (diluted from Formal-Fixx, 9990244, ThermoFisher Scientific) and 0.01% glutaraldehyde in Tris-buffered saline for 15 minutes when probing α-synuclein (Lee & Kamitani, 2011; Miner et al., 2023; Sasaki et al., 2015).

As a loading control, Total Protein staining was performed with the REVERT stain (926-11010, LI-COR). Briefly, membranes were exposed to REVERT diluted 1:5 in methanol for 1.5 minutes, and then washed twice for 30 seconds with 6.7% glacial acetic acid and 30% methanol in water. The REVERT stain was fully removed by incubating in LI-COR REVERT Destaining solution (926-11013, LI-COR) for 11 minutes, prior to blocking with 100% LI-COR Intercept (TBS) blocking solution (927-60003, LI-COR). Immunostaining for the target proteins was carried out using primary and secondary antibodies, diluted in three parts LI-COR Intercept (TBS) Blocking Buffer (927-60003, LI-COR) in one part 10 mM Tris-buffered saline, with 0.1% Tween-20. Primary and secondary immunoblotting antibodies are listed in Table 1 and Table 2. Highly cross-adsorbed antibodies from Invitrogen and Jackson Laboratories were used for immunoblotting at 0.133 μg/mL and 0.05 μg/mL, respectively. The blots were imaged using the Odyssey Classic or the Odyssey M imager and quantified with LI-COR Image Studio.

For the in vivo studies, insoluble α-synuclein was fractionated as previously published (Bhatia et al., 2023; Miner et al., 2023; Volpicelli-Daley, Luk, et al., 2014), from pooled piriform cortex and amygdala samples, as sample volumes<800 μL cannot withstand the g forces in our ultracentrifuge (MX 150, Sorvall Inc.). Briefly, tissues were probe-sonicated (Model XL2020, Misonix Inc.) in 1% Triton X-100-containing Cell Lysis Buffer (9803S, Cell Signaling Technologies). The cell lysis buffer was further supplemented with 1% protease inhibitor cocktail (P8340, Sigma-Aldrich), 1 mM phenylmethylsulfonylfluoride (PMSF; P7626, Sigma-Aldrich), and 1% protease inhibitor cocktail (2 mM imidazole, 1 mM sodium fluoride, and 1 mM sodium orthovanadate). A fixed-angle rotor (S55-A2, Sorvall Inc.) was subjected to the highest acceleration and deceleration forces (values of 9 on the Sorvall MX 150), and samples were ultracentrifuged at 100,000×g at 4° C. for 30 mins. Next, the Triton X-100-soluble supernatant was collected without disturbing the Triton X-100-insoluble pellet. The insoluble pellet was then resuspended in the same buffer as above and ultracentrifuged. The supernatant was collected as the Triton X-100 “wash” fraction. The second pellet (i.e., the fraction insoluble in the nonionic detergent) was then resuspended by sonication with the aforementioned probe in 2% sodium dodecyl sulfate in Tris-buffered saline. Protein concentrations were determined by the NanoDrop One^{C} (NanoDrop One C, ThermoFisher Scientific).

### Pharmacokinetic Analyses

Pharmacokinetic analyses were completed for AG161-41 and AG161-47 at PharmaDirections by intravenous and oral cassette administration (n=9 male CD-1 mice per compound; Pharmaron Study Protocol PH-DMPK-PDS-21-001; note that AG161-47 is named AGN11 and AG161-41 is named AGN5 in the Supplemental Material). Briefly, each compound was administered through intravenous and oral routes, and plasma samplings were collected from 2 to 24 hours post-injection for analyses by high-performance liquid chromatography and mass spectrometry (Prominence (Degasser DGU-20A5R(C), Serial NO. L20705619752 IX; Liquid Chromatograph LC-30AD Serial NO. L20555611120 AE and L20555611116 AE; Communications Bus Module CBM-20A, Serial NO. L20235635001 CD, Auto SIL-20AC HT, Serial No. L20355305356 AE; Rack changer II, Serial No. L20585300757 SS; AB Sciex Triple Quan 5500 LC/MS/MS instrument (Serial NO. EF20711807)). A HALO 90A, C18 2.7 μm 2.1×50 mm USKL004340 column was used and the mobile phase was 5 or 95% acetonitrile in water with 0.1% formic acid (see Supplemental Material).

### Tablet Preparation

All materials were ground in a mortar or milled to produce particles below 250 microns in diameter. Microcrystalline cellulose (Avicel® PH101, FMC Biopolymer) was mixed with AG161-47 in a mortar and pestle using geometric dilution prior to blending with carbohydrates, fats, proteins, vitamins, and minerals. Addition of AG161-47 in the active formulations was offset by a corresponding reduction in the amount of microcrystalline cellulose. Larger scale placebo formulations were compressed on a HT-AP38-MSU tablet press (Elizabeth HATA), while drug-containing formulations were compressed on a Presster tablet press (Metropolitan Computing Corp., now Natoli Engineering). All tablets were formed using standard B tooling (9.5 mm, concave) to produce tablets nominally weighing 400 mg each. Tablet doses were calculated using the following equation: Weight of AG161-47 added to each tablet (mg)=desired dose (mg of drug/1000 g mouse bodyweight)×50 g as the average weight of CD-1 male mice at 11 months of age (Table 3). The weight of AG161-47 for each tablet (0.25 mg for the 5 mg/kg tablet and 0.50 mg for the 10 mg/kg tablet) was multiplied by the total number of tablets for each dose in the manufacturing process. The tensile strength of all tablets was approximately 0.26 MPa, to retain tablet shapes until use, but could be readily chewed by the animals. All materials, including the tablets, were refrigerated at ˜8° C. prior to being placed in animal cages for consumption.

### Animals, Surgeries, and Behavior Testing

All animal procedures were approved by the IACUC at Duquesne University of The Holy Spirit and conducted in compliance with the NIH Guide for Care and Use of Laboratory Animals. Male CD-1 retired breeders were obtained from Envigo (Indianapolis, IN) and housed individually in a 12-hour light/dark cycle with ad libitum access to food and water, including during the tablet administration period. At 11 months of age, animals were exposed to one placebo tablet per day for two days prior to surgery. Drug or vehicle-containing tablets were then introduced in the home cage between 3-4 PM every day for 6 weeks, from the day of the stereotaxic surgery onwards. Previously administered tablets, if not fully consumed, were removed from the cages, without disturbing the bedding or touching the animals. Please note that CD-1 IGS mice are a genetically outbred, robust stock included here to increase genetic diversity (Aldinger et al., 2009; Hsieh et al., 2017). They are not inbred and isogenic like C57BL/6J mice and completely distinct from CD1^{-/-} mice. The latter is a mouse line, not a mouse stock, and never used by us.

Stereotaxic infusions of PBS or PFFs were made in the murine OB/AON as previously published (Bhatia et al., 2021; Mason et al., 2016; Miner et al., 2022). Briefly, animals were anesthetized using 3-4% vaporized isoflurane and heads were stabilized in a stereotaxic frame (Stoelting, IL). The coordinates used for infusions were verified as AP+4.0 mm, ML−1.0 mm, and DV−2.5 mm relative to Bregma (from the top of the skull) by injecting blue food dye in spare mice of the same age and cohort. Bilateral infusions of 5 μg of fibrils or an equivalent volume of phosphate-buffered saline (PBS; 1 μL) were made with a Hamilton syringe (80330, Hamilton, Reno, NV) using a motorized pump (Stoelting), at a rate of 0.250 μL per minute. The needle was slowly retracted from the brain after a 3 to 4-minute rest period. Animals were administered 0.05 mg/kg Buprenex subcutaneously before recovery from the isoflurane anesthesia, and Lidocaine was applied to the incision area for three subsequent days.

At the end of six weeks of daily tablet delivery, the open field test was used to gauge locomotion in an uncovered arena, as described (Miner et al., 2022). A 5-minute trial was completed in a 2 ft.×2 ft.×1.5 ft. (L×W×H) arena divided into 16 squares (6 in.×6 in.; 12 peripheral and 4 central squares). Each trial was commenced by placing a mouse at the center of the arena and the animal was allowed to roam freely for 5 minutes. The walls and floor were cleaned between trials with Quatricide (Pharmacal Research Labs) followed by water. A blinded observer recorded the number of rears and ANY-maze software (Stoelting) was used to track movement paths, the number of central square entries, distance traveled in meters (m), mean and max speeds (m/s), the number of immobile episodes (number of episodes during which the animal stopped traveling across the floor), and the time inactive in seconds (s) (total time spent motionless).

The sucrose preference test was used to evaluate the animals' preference for 1% sucrose-sweetened versus regular drinking water (Miner et al., 2022). Mice were individually housed with ad libitum access to two randomly placed bottles of regular water and sucrose-sweetened water over a 24-hour period. Sucrose water preference was calculated as relative intake of the 1% sucrose solution using the following equation: [1% sucrose solution consumed in grams/(1% sucrose solution consumed+regular drinking water consumed in grams)]×100. Total liquid intake was calculated using the following equation: [1% sucrose solution (g)+regular drinking water (g)]/body weight (g). The novel odor habituation test, Y-maze test, and marble burying tests were also completed as described (Miner et al., 2022), but showed no impact of AG161-47.

After six weeks of daily exposure to a single tablet, animals were sacrificed with exposure to 5% vaporized isoflurane followed by decapitation. The OB/AON, amygdalae, piriform cortices, entorhinal cortices, and hippocampi were dissected from the right hemisphere and frozen on dry ice. The left hemispheres were immersion-fixed in 4% formaldehyde (Formal-Fixx, 9990244, ThermoFisher Scientific) in 10 mM PBS for 10 days. Next, brains were immersed in 30% sucrose in 10 mM PBS for one week prior to sectioning on a freezing microtome (Model 860, Rankin, Holly, MI). Forty micron-thick sagittal sections were stored in cryoprotectant at −20° C. as a 1-in-5 series, until immunostaining procedures.

### Tyramide Signal Amplification

A 1-in-5 series of sagittal mouse brain sections was permeabilized using 0.5% Triton-X 100 in 10 mM PBS. Endogenous peroxidases were quenched for 15 minutes using 0.3% H₂O₂ (H325-100, ThermoFisher) in 10 mM PBS. Sections were then blocked using Li-COR Intercept PBS blocking buffer for one hour (927-70001, Li-COR). Next, sections were incubated overnight in rabbit primary antibodies against pSer129 and guinea-pig primary antibodies NeuN in the same blocking solution. After primary antibody incubations, sections were incubated for one hour in horseradish peroxidase-conjugated donkey anti-rabbit secondary antibodies at 0.8 mg/mL (711-035-152, Jackson Laboratories) for amplification of the pSer129⁺ signal. A 1:500 dilution of the CF dye 543 (92172, Biotium) was prepared with 0.0015% H₂O₂ in the amplification buffer (99832, Biotium). The sections were then incubated with the Hoechst 33258 pan-nuclear stain and secondary donkey anti-guinea pig antibodies for one hour (Tables 1 and 2). Stained sections were mounted, coverslipped, and imaged on an epifluorescence microscope (Olympus IX73, B&B Microscopes, Pittsburgh, PA).

### Artificial Intelligence/Deep Learning-Assisted Image Analyses

The rostral and medial piriform cortex was defined as described on Sagittal Plates 114 to 121 of Franklin and Paxinos' stereotaxic mouse brain atlas (Franklin & Paxinos, 2013). This area was chosen as it is present on more mouse brain sections when cut in the sagittal plane, compared to the AON or cortical amygdala. Photomicrographs centered on this anterior piriform subregion (immediately caudal to the AON) were captured in 3 to 5 sections per animal on an Olympus epifluorescence microscope (Olympus IX73, B&B Microscopes, Pittsburgh, PA) and analyses of pSer129⁺, Hoechst⁺, and NeuN⁺ structures were completed using the Generalized Network algorithm in the TruAI™ Deep Learning module (Olympus cellSens Dimension, Desktop Version 3.2), as previously described (Bhatia et al., 2023). The Ground Truth of the Neural Network was defined by at least 70,000 iterations as positive signal, denoted by the training labels. The training labels were defined for each target stain on five regions in each of 5-6 randomly chosen images by a blinded observer, through a combination of manual and threshold-based methods. The built-in batch-processing macro functionality of the Olympus cellSens Dimension Desktop Version 3.2 was then used to fully automate the Neural Network analysis of all images for each label. The following measurements were automated: 1) Counts, 2) mean grayscale intensity (MGI), 3) mean surface area of individual structures (defined as size), 4) the area in pixels occupied by the structures as a fraction of the total area of the image (defined as area fraction), 5) the area of the target relative to the area of a circle with equal perimeter (defined as shape factor), 6) the mean perimeter of individual structures, and 7) the distance to the nearest neighbor (the spatial dispersal of the target structures).

### Human Postmortem Tissues

Human tissues from the OB and amygdala were obtained from the Human Brain and Spinal Fluid Resource Center at the West Los Angeles VA and the University of Miami Brain Endowment Bank, through the Institutional Review Board-approved repository at the National Institutes of Health NeuroBioBank. Samples from six women and seven men diagnosed with Lewy body disorders had been harvested postmortem by board-certified neuropathologists. Age-matched OB and amygdala tissues from six women and seven men unaffected by Lewy body disorders were also harvested as negative controls. All samples were deidentified prior to shipping and not subject to reapproval at Duquesne University of The Holy Spirit according to the IRB Chair. Tables 4 and 5 list demographic information, which can also be viewed online on the NeuroBioBank web portal. We found no group differences for age or postmortem intervals (Bhatia et al., 2021). Images of the amygdala dissections are published (Miner et al., 2022). Human samples were subjected to the same immunoblotting procedures as detailed above.

### Statistical Analyses

All statistical testing was two-tailed with an alpha threshold of 0.0500 and performed in GraphPad Prism (Version 9.5.1). Depending on data distributions, the parametric paired t test or the nonparametric Mann-Whitney U test was used for comparisons between two groups. For comparisons of more than two groups, the appropriate ANOVA (one, two, or three-way) was followed by the Bonferroni post hoc test for multiplicity-adjusted pairwise comparisons. Downstream statistical tests (i.e., parametric vs. non-parametric) were chosen a priori based on the results of the Shapiro-Wilk normality test. Non-Gaussian data were log₁₀ transformed, or if there were zero values, data were square-root transformed, as indicated in figures and legends (note that all datapoints are shown before and after transformation). Data points from the same in vitro experiment (i.e., with cells harvested from the same set of rat pups and run side-by-side on cell culture plates) were matched for all comparisons across rows, columns, and subcolumns. Thus, a repeated measures ANOVA (generalized linear model) was applied for the in vitro results, unless there were missing values from an imbalanced n, in which case a mixed-effects repeated measures model was used. For the in vivo results, regular ANOVAs were used, without any matching. The Shapiro-Wilk test was used to test the normality of data for correlation tests. We report Pearson r values for normally distributed data without outliers and Spearman r values for the rest. Pearson or Spearman r values of 0.4000 and two-tailed p values≤0.0500 were used as statistical thresholds. Best-fit lines and 95% confidence intervals were added from linear regressions only to illustrate the slopes of potentially linear correlations, with Lewy-like pathology measures placed on the X-axes. No statistical outliers were excluded from analyses or data presentation within graphs. Analyses were blinded wherever possible, except for SDS-PAGE, where samples were not blind-coded but loaded in a predetermined order onto the gels. Blots are shown at full length to show all potentially nonspecific antibody binding.

### Results

Molecular Modeling: We report that compounds AG161-41 and AG161-47 exert dual effects on microtubules, with stabilizing as well as destabilizing effects. We also designed analogs of AG161-41 and AG161-47 with potential modulatory effects on microtubules, by exploring changes at the N⁴-position. Substituting the N⁴-phenyl ring of AG161-47 with 5,6,7,8-tetrahydronaphthalen-2-yl and benzofuran-5-yl substituents added both bulk and lipophilic character. These changes provide steric hinderance to the tricyclic compounds and hamper binding to the colchicine site on microtubules, thereby impairing depolymerizing potential. These potential effects were supported by the molecular docking study that was performed. The tricyclic scaffold of AG161-47 lies in the pocket lined with Alaβ316, Alaβ317, Ileβ318, Valβ238, Cysβ241, Leuβ248, Alaβ250, Aspβ251 and Leuβ255, and the 9-NH forms a hydrogen bond with the backbone carbonyl of Valβ238. The N⁴-phenyl ring makes hydrophobic interactions with Alaα180, Alaβ316, Lysβ352, Leuβ255, Asnβ258 and Metβ259, while the N⁴-methyl group interacts with Leuβ248 and Alaβ354. When docked, compounds AG182-126 and AG185-174 are unable to achieve a similar binding pose within the colchicine site. The molecules therefore flip to accommodate the bulkier N⁴-substitution. Superimposition of these flipped poses within the binding site of docked AG161-47 shows a steric clash between the scaffolds of AG182-126 and AG185-174 and Lysβ352. Additionally, the benzofuran ring of AG182-126 clashes with Ileβ378, while the 5,6,7,8-tetrahydronaphthalene ring of AG185-174 clashes with Cysβ241. This hindrance in binding of AG182-126 and AG185-174 at the colchicine site is hypothesized to result in the desired mild inhibition of tubulin assembly, akin to that observed with AG161-47 in molecular modeling.

To understand the microtubule stabilizing effects of AG161-47, molecular modeling studies were performed in the vinca binding site of tubulin. The N⁴-phenyl substituent is engaged in π-πstacking interactions with Tyr210 and Tyr 224 of β1-tubulin. The 2-NH₂ makes a H-bond with Ser174. The docked AG161-47 also caused a ˜8.5 Å inward shift of the α-helix of α2-tubulin compared to vinblastine-bound tubulin, bringing it closer to the β1-tubulin monomer and resulting in less-curved microtubule assembly, thus precluding microtubule disintegration and promoting mild microtubule stabilization, similar to previously reported triazolo pyrimidine compounds (Saez-Calvo et al., 2017). AG161-41, AG182-126, and AG185-174 were also docked in the vinca binding site of tubulin (PDB: 5NJH) and caused a similar in silico α-helical shift.

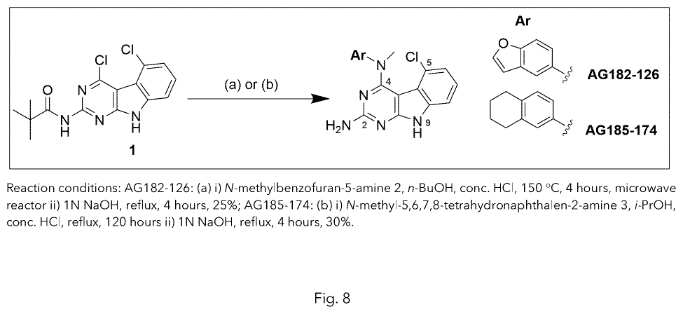

Syntheses: Compounds AG161-41 and AG161-47 were synthesized. The synthesis of compounds AG182-126 and AG185-174, intermediate 1 was obtained by following the reported synthetic procedure (Gangjee et al., 2010; Islam et al., 2021). Displacement of the 4-Cl of 1 with the corresponding, appropriately substituted N-methyl anilines followed by base-mediated deprotection of the 2-NH₂ group yielded the target compounds AG182-126 and AG185-174 (see FIG. 8).

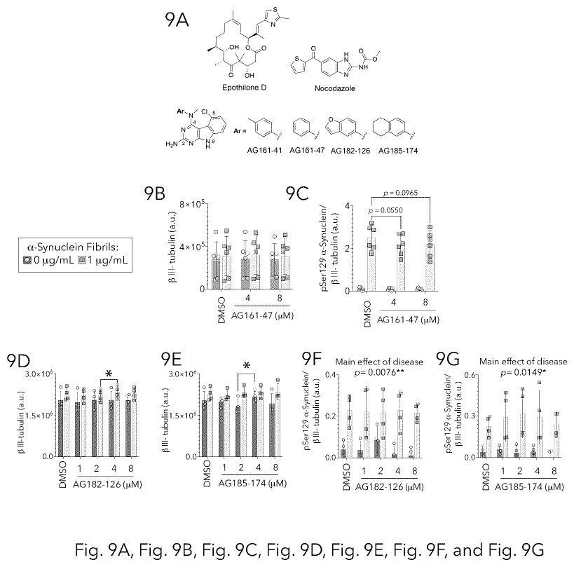

AG161-47 reduces α-synucleinopathy in primary hippocampal cultures: Primary hippocampal cultures were generated from postnatal rat pups and challenged with preformed α-synuclein fibrils at 1 μg/mL for 10 days in vitro (FIG. 9B). Cultures were simultaneously treated with escalating concentrations of the microtubule-targeting compounds. Previously, we showed that preformed fibrils generate dense α-synucleinopathic inclusions in primary hippocampal neurons (Bhatia et al., 2021; Miner et al., 2022), as defined by hyperphosphorylated α-synuclein (pSer129), the best available marker for Lewy bodies and Lewy neurites (Delic et al., 2018; Lashuel et al., 2022), as well as the neuronal somatodendritic marker microtubule-associated protein 2 (MAP2) and the pan-neuronal microtubule marker βIII-tubulin. Although the phosphorylation of α-synuclein at the serine 129 residue has physiological roles under baseline conditions (Parra-Rivas et al., 2022; Ramalingam et al., 2023), there is a marked increase in this measure in Lewy body disorders (Anderson et al., 2006; Delic et al., 2018; Hasegawa et al., 2002; Killinger et al., 2023; Polinski et al., 2018). We also previously demonstrated that α-synuclein fibrils do not generate severe cell loss in our mixed-sex postnatal hippocampal cultures within 10 to 14 days of exposure. The preformed fibril model was used here to model early-stage Lewy-related pathology—without severe neurodegeneration in vitro.

AG161-47 is shown to be a dual-acting microtubule modulator, with simultaneous polymerizing and depolymerizing effects on microtubules at relatively high concentrations when examined with immunocytochemistry in HeLa cells, and polymerizing effects in an in vitro test of porcine brain tubulin assembly. When we tested the influence of AG161-47 on pSer129⁺ α-synucleinopathy by In-Cell Western analyses, a decrease in pSer129 levels was observed, with no change in neuronal viability, as measured by expression of the neuron marker βIII-tubulin (FIGS. 9B-C). The two additional heterocyclic compounds, AG182-126 and AG185-174, designed to serve as dual-acting microtubule modulators were tested but exerted no consistent effects on neuronal viability (FIGS. 9D-E) or on pSer129⁺ α-synucleinopathy (FIGS. 9F-G). The microtubule stabilizer epothilone was found to be toxic to primary hippocampal cultures at nanomolar concentrations and therefore could not be employed as a positive control (data not shown).

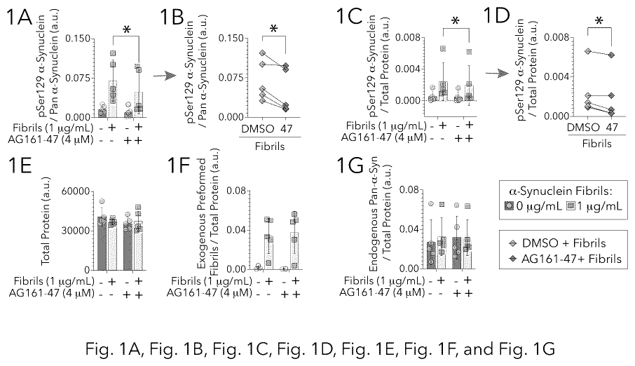

We also assessed pSer129 levels by SDS-PAGE and immunoblotting (FIGS. 1A-G). Note that monomeric endogenous α-synuclein migrates through denaturing gels at ˜17 kDa (Sharon et al., 2001), whereas the denatured fibrils migrate at ˜14 kDa, the predicted mass of recombinant α-synuclein monomers. The exogenous recombinant fibrils show no evidence of phosphorylation in the literature (Volpicelli-Daley, Luk, et al., 2014; Volpicelli-Daley et al., 2011). As expected, the levels of exogenous and endogenous pan α-synuclein levels were not affected by AG161-47, but the hyperphosphorylation of α-synuclein was lowered by 4 μM AG161-47 (FIGS. 1A-G). Other measures, such as changes in pSer129 as a function of total protein concentrations (FIGS. 1C-D), total protein concentrations in the cell lysates (FIG. 1E), levels of denatured, exogenous α-synuclein (FIG. 1F) and the levels of endogenous pan α-synuclein were modified by AG161-47 (FIGS. 1C-G). It is to be noted that highly phosphorylated and abnormally aggregated α-synuclein is a major component of Lewy bodies found in patients with PD. (Kawahata et al., 2022)

The microtubule destabilizer/depolymerizer and kinase inhibitor nocodazole blocks AG161-47 activity: We qualitatively compared AG161-47 with the established microtubule destabilizer/depolymerizer nocodazole (Baas & Black, 1990; Baas et al., 2016; Bates & Eastman, 2017; Kapellos et al., 2016; Koenigsknecht & Landreth, 2004; Kuhn, 1998; Mandrekar et al., 2009; Nakayama et al., 2012; Park et al., 2012), using a nocodazole shock treatment followed by cold methanol fixation in primary hippocampal cultures, as previously described (Ferreira et al., 2018; Warren et al., 2020). Primary hippocampal cultures were treated with vehicle or AG161-47 at 8 μM or nocodazole at 50 nM and 5 μM. AG161-47 did not alter the appearance of βIII-tubulin⁺ structures in primary hippocampal neurons relative to treatment with vehicle. As expected, the nocodazole-treated groups displayed evidence of neurotoxicity and severe beading of axonal microtubules. AG161-47 does not.

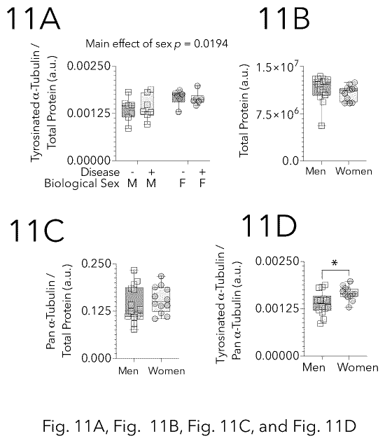

FIGS. 11A-D show the impact of biological sex on tyrosinated α-tubulin in the human amygdala. Amygdala samples harvested postmortem from individuals diagnosed with or without Lewy body disorders were subjected to SDS-PAGE and immunoblotting for detyrosinated, acetylated, or tyrosinated (FIG. 11A) α-tubulin. The REVERT Total Protein Stain was employed as the loading control. Only the tyrosinated α-tubulin blots are shown, as the other antibodies were subpar on amygdala tissues. As no statistically significant effect of Lewy body disease on tyrosinated α-tubulin was observed, all data were collapsed in FIGS. 11B-D to focus on the effect of sex with higher power. Full-length blots were established (not shown). The n is 12 women and 13 men, and data in FIG. 11A were analyzed by two-way ANOVA/Bonferroni, and the remaining panels show **p≤0.0100 by a two-tailed, unpaired t-test.

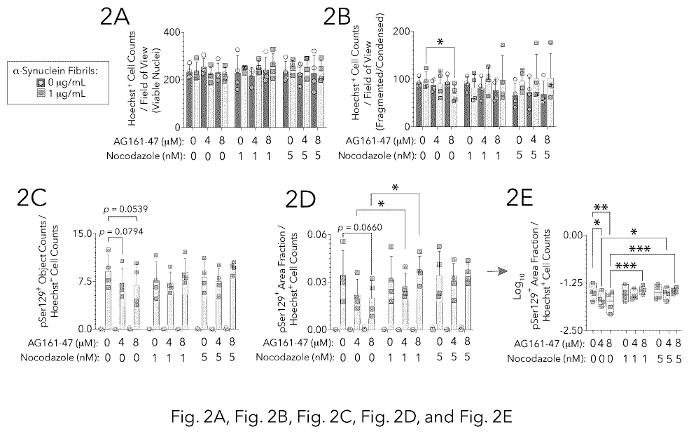

We then tested if sublethal concentrations of nocodazole would reduce or abolish the activity of AG161-47 on pSer129⁺ structures (defined here as “inclusions”), to determine if AG161-47 was affecting microtubules as one of its purported mechanisms of action. Primary hippocampal cultures were treated with preformed fibrils and escalating concentrations of AG161-47 or sublethal concentrations of nocodazole (1 and 5 nM). We observed no statistically significant changes in the number of viable Hoechst⁺ cells in any treatment group. FIG. 2A shows data that are consistent with our earlier work on the lack of robust toxicity of preformed fibrils in primary hippocampal cultures and the lack of impact of AG161-47 on βIII-tubulin levels in the In-Cell Western analyses. However, at the highest concentration (8 μM), AG161-47 reduced fragmented and condensed Hoechst⁺ nuclei in the fibril-treated group. The moderating impact of AG161-47 on fragmented/condensed cell counts in the fibril groups was prevented by both concentrations of nocodazole. Higher concentrations of AG161-47 were not more effective and began to elicit evidence of cell loss in preliminary work.

We also assessed whether the ability of AG161-47 to reduce pSer129⁺ α-synucleinopathy in primary neurons would be prevented by nocodazole (FIG. 2C-E). As observed in the pSer129 In-Cell Western analyses of FIG. 9C, only modest trends towards reductions were observed in the raw pSer129⁺ object counts per cell, but the log₁₀-transformation of the non-Gaussian area fraction measurements revealed that nocodazole abolished the activity of AG161-47 against pSer129⁺ α-synucleinopathic inclusion load (FIG. 2C-E). These observations support the idea that AG161-47 reduces α-synucleinopathic inclusion load via direct or indirect action on microtubule dynamics, although it is worth noting that all pharmacologic tools have off-target effects, and nocodazole is no exception with known inhibition of kinases involved in phosphorylation of α-synuclein. (Park et al., 2012)

AG161-41 also reduces α-synucleinopathy in primary hippocampal cultures: As proof-of-principle, we tested the other Janus-faced compound, AG161-41, shown to accelerate the temporal kinetics of porcine brain tubulin in vitro assembly compared to DMSO, but not to raise tubulin polymerization to the same levels as paclitaxel. In primary hippocampal cultures, AG161-41 reduced the total area occupied by the pSer129⁺ objects per cell and the average pSer129⁺ object size per cell were also reduced by exposure to 4 μM AG161-41 (FIG. 3B-C). Here, the vehicle control elicited small increases in inclusion size, and the reductions in Lewy-related pathology by AG161-41 were recorded (FIG. 3C).

We then confirmed that the pSer129 α-synuclein⁺ objects were insoluble in the nonionic detergent Triton X-100, in the presence or absence of AG161-41 and AG161-47. Thus, Triton X-100 was added simultaneously with formaldehyde prior to immunostaining with rabbit monoclonal antibodies against pSer129. As expected, the pSer129⁺ inclusions were indeed insoluble in Triton X-100.

AG161-47 impacts microtubule stabilization markers but negates the effects of nocodazole: We have shown that AG161-47 is not a powerful destabilizer of the microtubule network in neurons compared to nocodazole, but that it reduced nuclear fragmentation and condensation and the area occupied by pSer129⁺ objects in a nocodazole-sensitive manner. To further evaluate how AG161-47 affects microtubules, we used established markers of microtubule stabilization (detyrosination) and lack of stabilization (tyrosination) (Akhmanova & Kapitein, 2022; Arce et al., 1975; Baas et al., 2016; Gundersen et al., 1984; Janke & Bulinski, 2011; Mitchison & Kirschner, 1984; Portran et al., 2017; Zhou et al., 2019). Detyrosination may not impart cytoskeletal stability per se but the probability of this post-translational modification rises in parallel with increased microtubule lifespan and is therefore naturally higher in the stable microtubule fraction (Baas et al., 2016; Gundersen et al., 1984; Janke & Bulinski, 2011). Conversely, non-detyrosinated (i.e., tyrosinated) tubulin polymers are more commonly encountered within the non-stabilized fraction of neuronal microtubules (Gundersen et al., 1984; Janke & Bulinski, 2011).

Western immunoblotting for markers of post-translational modifications was performed on cell extracts of primary hippocampal cultures treated with fibrils, AG161-47, and nocodazole. Although there were no changes in mean values, AG161-47 raised the inter-experimental variability of levels of detyrosinated (stable) α-tubulin (FIGS. 4A-B). AG161-47 also exerted no consistent effects on tyrosinated (nonstable) α-tubulin (FIGS. 4C-D). Similarly, no effects of the three treatments were noted when the stability markers were expressed as a fraction of the total protein stain (not shown), which reflects the total mass of α-tubulin with the post-translational modification in question, as a fraction of total protein concentrations within the cell extracts.

Another post-translational modification more commonly observed in the stable fraction of microtubules is the acetylation of intraluminal lysine 40 (K40) of α-tubulin, catalyzed by α-tubulin N-acetyltransferase (ATAT1) (Baas et al., 2016; Janke & Bulinski, 2011; L'Hernault & Rosenbaum, 1985; Nekooki-Machida & Hagiwara, 2020; Portran et al., 2017; Xu et al., 2017). Acetylation has been argued to impart microtubules with mechanical resistance, as ATAT1 depletion raises the probability of microtubule breakage (Naren et al., 2023; Portran et al., 2017; Xu et al., 2017). Unexpectedly, we observed that 5 nM nocodazole raised the levels of acetylated (stable) α-tubulin (FIGS. 4E-F), suggesting that at low concentrations of this agent, microtubules display evidence of rigidity in the presence of fibrils. AG161-47 abolished this nocodazole-mediated increase in acetylated α-tubulin levels in the fibril-treated group (FIGS. 4E-F). This is most easily appreciated in the two-way ANOVA of FIG. 4F, where the simultaneous addition of nocodazole and AG161-47 canceled increases in microtubule acetylation in fibril-treated groups, and there was a statistical interaction between the two independent variables pulled out for the focused analysis (nocodazole vs. vehicle×AG161-47 vs. vehicle; FIG. 4F). When fibril-treated groups were subjected to a focused analysis under conditions of experimental Lewy body disease (in the presence of fibrils but the absence of nocodazole), acetylated α-tubulin levels were not higher with exposure to AG161-47, but the two-tailed p value was 0.0751 (see paired, two-tailed t test in right panel of FIG. 4G). These observations reveal the possibility of subtle microtubule-modifying effects of AG161-47 when nocodazole is not on board (FIG. 4G), but also that AG161-47 impedes the acetylating impact of nocodazole under fibril exposure conditions (FIG. 4F).

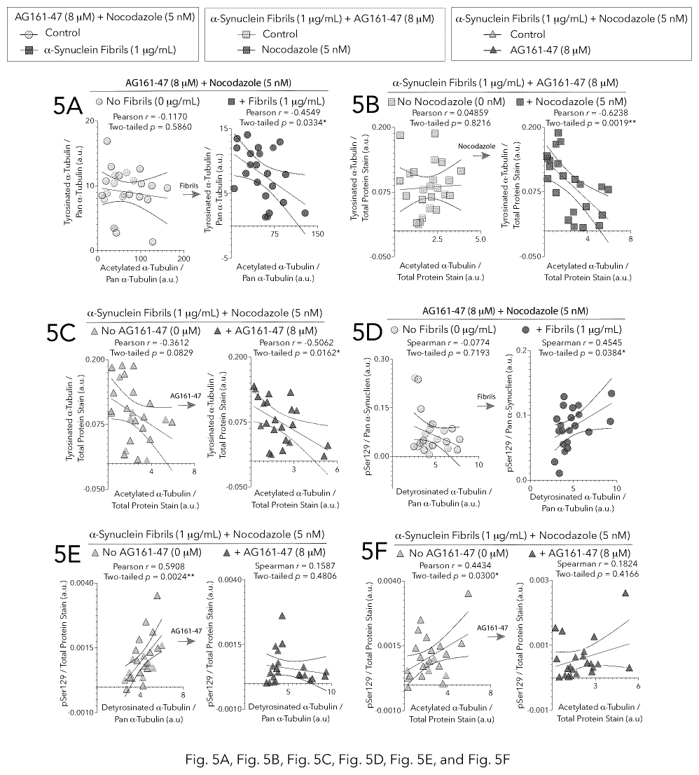

Exposure to fibrils, nocodazole, or AG161-47 exerts unique actions on α-synucleinopathy and microtubules: Next, we performed a correlation analysis of markers of microtubule stability in the presence vs. absence of fibrils, nocodazole, or AG161-47. We first observed that all three of these treatments elicited an inverse relationship between stable, acetylated α-tubulin and nonstable, tyrosinated α-tubulin (FIG. 5A-C). As described above, non-detyrosinated (or tyrosinated) tubulin polymers are more commonly encountered within the nonstabilized fraction of neuronal microtubules, whereas stable microtubules with longer lifespans have higher probabilities of being detyrosinated and acetylated. Fibril exposure forced the appearance of a negative correlation between the fractions of pan α-tubulin that were tyrosinated and acetylated (FIG. 5A), whereas nocodazole and AG161-47 elicited negative correlations for the entire masses of tyrosinated and acetylated α-tubulin (as a fraction of all proteins present in the extracts; FIG. 5B-C). Hence, despite a lack of robust changes in the ANOVAs on microtubule stability markers shown in FIGS. 4B-H, all three treatments appear to modify microtubules, with greater similarities between nocodazole and AG161-47 than observed between fibrils and AG161-47.

Compared to fibril or nocodazole exposure, AG161-47 exerted unique effects on the relationship between microtubule stabilization status and hyperphosphorylated α-synuclein (pSer129 levels). Unexpectedly, fibril treatments tended to elicit positive correlations between pSer129 and detyrosinated (stable) α-tubulin (FIG. 5D), whereas AG161-47 opposed these effects and abolished positive correlations between Lewy-related pathology and markers of microtubule stabilization (FIGS. 5E-F). These results suggest that higher levels of Lewy-related pathology are observed in vitro when tubulin heteropolymers are more rigid or there is a greater mass of rigid tubulin heteropolymers—unless AG161-47 is on board, as it severs these associations. Hence, AG161-47 may shift microtubule dynamics directly or indirectly to oppose the impact of fibrils on cytoskeletal rigidity.

AG161-47 mitigates formation of insoluble, hyperphosphorylated α-synuclein in an in vivo model of limbic Lewy body disease: As AG161-47 and AG161-41 were the only effective compounds of the tricyclic analogs we tested, we evaluated their pharmacokinetic properties in male CD-1 mice (AG161-41 is AGN5 (4+1=5) and AG161-47 is AGN11 (4+7=11). During this work, no abnormal clinical symptoms were observed (i.e., no evidence of changes in body temperature, convulsions, symptoms of lassitude or hyperactivity, abnormal posture, changes in breathing rate, body weight loss, urine or fecal changes, coat condition, or mortality). AG161-41 was tagged as displaying slightly more desirable pharmacokinetic properties than AG161-47, but AG161-47 displayed better bioavailability than AG161-41 and was also more effective in lowering Lewy-related pathologies in the primary hippocampal culture assays. Therefore, AG161-47 was chosen over AG161-41 for subsequent in vivo work.

We tested the therapeutic potential of AG161-47 in an animal model of limbic-centered, early-stage Lewy body disorders. Similar to the in vitro model, we sought to avoid massive, irreversible neurodegeneration in our in vivo model (Bhatia et al., 2021; Bhatia et al., 2023; Mason et al., 2016; Mason et al., 2019; Miner et al., 2023; Miner et al., 2022; Nouraei et al., 2018). During the animal study, we also developed a new oral delivery platform that avoids drug dissolution in water or any application of heat, thereby mitigating potential drug degradation. We were able to compress AG161-47 with carbohydrate, fat, and protein nutrients and a cellulose excipient into palatable tablets with the appropriate chewable consistency for voluntary rodent consumption.

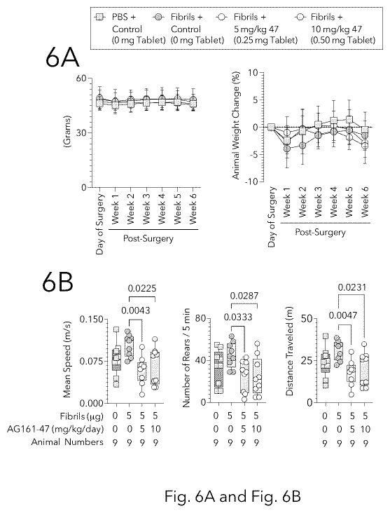

Animals infused bilaterally with preformed fibrils in the OB/AON were exposed to one tablet per day with 0 (placebo), 0.25, or 0.50 mg AG161-47, every day for the next six weeks, starting at 11 months of age (n=9 mice per group), without removal of access to regular food and water. With this design, we targeted achieving ˜5 or 10 mg AG161-47 per kg bodyweight, as shown in Table 3. Although there was some variation in the dose ingested per unit bodyweight, we add that the dose of tablets is usually fixed in clinical studies—regardless of human body mass. In a heatmap (not shown), 26 out of 36 mice consumed all tablets every day following day 1 of exposure, over the course of six sequential weeks. Thus, 72% of mice consumed 100% of these tablets after day 1, and 89% of mice consumed 80% or more of tablets after day 1. No loss of bodyweight was elicited by exposure to AG161-47 compared to the placebo group (FIG. 6A). Exposure to AG161-47 reduced locomotor activity in an open arena six weeks after fibril infusions (FIG. 6B), but no other behavior measures were impacted.

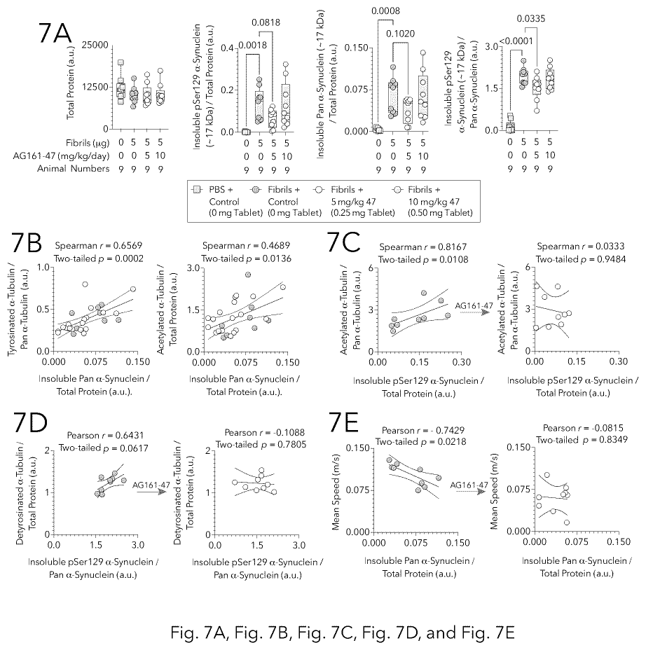

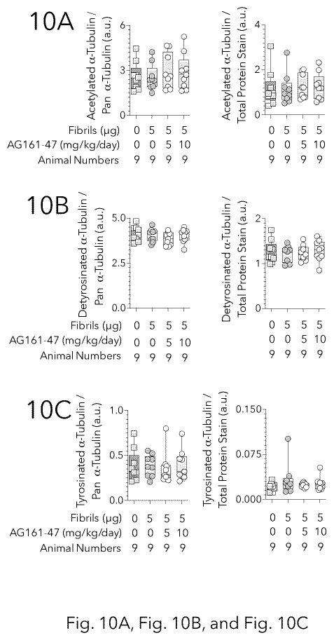

Next, the piriform cortex and amygdala from the other brain hemisphere were collapsed to generate sufficient tissues for nonionic detergent-based fractionation and ultracentrifugation at 100,000×g. In these tissue extracts, the 5 mg/kg dose of AG161-47 reduced the hyperphosphorylation of insoluble α-synuclein, without affecting total protein concentrations (FIG. 7A). Consistent with the in vitro observations on whole-cell extracts, the insoluble α-synuclein from in vivo tissue extracts migrated at ˜17 kDa, as expected (Sharon et al., 2001). On the other hand, AG161-47 exerted virtually no net effect on acetylated, detyrosinated, or tyrosinated α-tubulin levels in the whole-fraction OB/AON by immunoblotting (FIGS. 10A-C).

Finally, we performed a correlation analysis of the in vivo data. In fibril-treated mice, insoluble pan α-synuclein levels were in proportion to the (nonstable) fraction of α-tubulin that was tyrosinated, but a positive correlation was also observed with the acetylated (stable) fraction of α-tubulin (FIG. 7B). In fibril-exposed mice treated with placebo tablets, we observed a positive correlation between levels of insoluble pSer129 α-synuclein and the fraction of α-tubulin that was acetylated (FIG. 7C). Similar patterns were noted for the total mass of detyrosinated (stable) α-tubulin (FIG. 7D). In addition, the more insoluble α-synuclein was present, the slower the animals traveled in the open field test (FIG. 7E). Notably, none of these correlation patterns were evident in the presence of AG161-47 (FIG. 7C-E). Hence, greater α-synucleinopathic load may be associated with higher levels of acetylated (stable) α-tubulin in vivo, but this potentially causal relationship is uncoupled upon exposure to AG161-47. AG161-47 also uncoupled the inverse correlation between insoluble α-synuclein and locomotor speed. These findings suggest that AG161-47 opposes the links between microtubule stabilization status and α-synucleinopathy or neurological function in an experimental model of limbic Lewy body disease.

### Discussion

The present invention describes effects of dual-acting microtubule stabilizers/destabilizers on α-synucleinopathy in vitro and in vivo. The activities of AG161-47 and AG161-41 provide the discovery of a new pyrimido[4,5-b]indole scaffold for further evaluation in preclinical models as a prelude to clinical trials of Lewy body disorder. AG161-47 reduced levels of hyperphosphorylated α-synuclein at concentrations or doses that did not elicit frank cell loss in vitro or in vivo. However, the in vivo impact of AG161-47 was potentially U-shaped, pointing to dual effects on microtubule function and hence, no net change at the higher dose.

Although relatively high concentrations of AG161-41 and AG161-47 were needed to reduce in vitro Lewy-related pathology, it is important to note that this was by design, as highly potent microtubule modulators tend to be toxic.

In addition to α-tubulin modulation, we have also discovered that compounds AG161-41 and AG161-47 have inhibitory activities against eight of the kinases involved in the phosphorylation of α-synuclein and play diverse roles in Lewy body disorders (Table 6). Molecular modeling corroborates the activities observed in Table 6. Total inhibition of these kinases would be detrimental to neuronal viability. As such, mild inhibition (as accomplished by AG161-41 and AG161-47) is highly desirable. These results substantiate the multitargeted nature of the analogs.

AG161-41 and AG161-47 (previously identified as compounds 2 and 3, respectively) are dual-acting agents that shift microtubule assembly dynamics in spatiotemporal ways. For example, AG161-47 relaxed microtubules in the periphery of HeLa cells while bundling microtubules at perinuclear locations. Of those compounds tested, AG161-47 was the only one to raise the initial rate of polymerization of purified, porcine brain tubulin at substoichiometric concentrations in vitro (albeit not to the degree of the microtubule stabilizer paclitaxel/Taxol). All the evidence to date indicates that the net impact of AG161-47 on microtubules is distinct from classic microtubule stabilizing or destabilizing agents (Rohena et al., 2016), as demonstrated here in primary hippocampal neurons vis-à-vis nocodazole and epothilone, and as demonstrated in vitro and in vivo by the lack of robust effects of AG161-47 on microtubules.

Lewy body disorders are a family of neurodegenerative conditions in which the protein α-synuclein aggregates into toxic fibrils and seeds the formation of proteinaceous inclusion bodies in vulnerable neurons. (Braak et al., 2006; Del Tredici & Braak, 2016; Goedert et al., 2013; Hawkes et al., 2010) Lewy body disorders include Parkinson's disease (PD), dementia with Lewy bodies (DLB), and Alzheimer's disease with Lewy bodies (ADLB). Neurodegenerative disorders are believed to be associated with loss of stability of the neuronal cytoskeleton, which is formed from lateral and longitudinal assemblies of α-tubulin and β-tubulin heteropolymers. (Brouhard & Rice, 2018; Gudimchuk & McIntosh, 2021; Janke & Magiera, 2020; Manka & Moores, 2018; Mazzetti et al., 2023) However, microtubule heteropolymers are a challenging therapeutic target in neurodegenerative disorders, as excessive rigidity imparted by microtubule stabilizers is toxic to brain cells, such as neurons and glia. Thus, careful titration of microtubule stabilization vs. destabilization with dual-acting microtubule modulators will be necessary to treat neurodegenerative conditions. The objective of our work is to identify microtubule modulators that strike this delicate balance and reduce Lewy-related pathologies in experimental models of Lewy body disease.

We show that Compound 3, defined here as AG161-47 or Compound 11, accelerated and amplified the net in vitro assembly of tubulin purified from porcine brain and elicited both stabilization and relaxation of microtubules. (Rohena et al., 2016) Thus, pyrimido[4,5-b]indole tricyclic compounds designed to exert microtubule modulating effects were tested in primary hippocampal cultures challenged with preformed α-synuclein fibrils, as an established experimental model of Lewy-related pathology. (Bhatia et al., 2021; Volpicelli-Daley, Luk, et al., 2014)

In our primary hippocampal culture model, we discovered that AG161-47 alleviated α-synucleinopathy and nuclear fragmentation without neurotoxicity. As proof of principle, the dual-acting tricyclic pyrimido[4,5-b]indole compound, AG161-41, also reduced Lewy-related pathology without neurotoxicity. In contrast, the bicyclic pyrazolo[4,3-d]pyrimidine-based microtubule destabilizer, AG177-359, was found to be toxic. The microtubule depolymerizer nocodazole and the microtubule stabilizer epothilone were also found to be toxic in primary hippocampal cultures, confirming that excessive stabilization and destabilization are both undesirable in experimental Lewy body disease. We observed that AG161-47 interfered with the impact of fibrils on microtubule acetylation, induction of Lewy-like pathology, and nuclear fragmentation, and these effects were abolished by cotreatment with the microtubule disruptor nocodazole. We therefore believe that microtubule modulators exert sufficiently subtle effects along the microtubule trails, avoiding excessive flexibility, rigidity, or catastrophic loss of the three-dimensional structure of polymeric tubulin.

FIG. 1A shows the functional impact of heterocyclic pyrimido[4,5-b]indoles, levels of pSer129 α-synuclein as a fraction of pan α-synuclein (FIGS. 1A and 1B), pSer129 as a fraction of total protein concentrations (FIGS. 1C-D), total protein levels (FIG. 1E), exogenously applied preformed fibrils (FIG. 1F), and endogenous α-synuclein (i.e., excluding the synthetic fibrils) (FIG. 1G). Shown are the mean±S.D. For FIGS. 1A-1G, n=5 independent cultures for all groups. Each culture was run in duplicate or triplicate wells. *p≤0.0500, repeated measures (matching for each independent culture) two-way ANOVA/Bonferroni or a two-tailed paired t test on the fibril-treated groups alone (follow arrows in A and C). Statistically significant effects of experimental Lewy body disease or fibril exposure were observed in FIGS. 1A, 1C, and 1F (p≤0.0500), but the statistical impact of preformed fibrils in multiplicity-adjusted pairwise comparisons was not added to avoid figure crowding. Primary hippocampal cultures were treated with vehicle, AG161-47 (8 μM) or the positive control nocodazole (50 nM) for 5 min prior to 15 minutes of cold methanol. Cultures were immunostained for somatodendritic neuron marker MAP2 (not shown) and neuronal microtubule marker βIII-tubulin (not shown). Nuclei were labeled with the Hoechst reagent (not shown). Note that microtubules become wavy after cold methanol exposure even under physiological conditions (not shown). Severe loss of axons and dendrites and beading of βIII-tubulin⁺ structures were observed after exposure to 0.05 μM nocodazole but not 8 μM AG161-47.

FIGS. 2A-E show that the microtubule destabilizer/depolymerizer nocodazole disrupts the activities of AG161-47. Primary rat hippocampal cultures were treated for 10 days in vitro with preformed fibrils, AG161-47, and nocodazole (or vehicle). Representative images (not shown) of hippocampal cultures stained with the pan-nuclear marker Hoechst and immunolabeled for hyperphosphorylated α-synuclein (pSer129) were observed. Viable Hoechst⁺ cell counts per field of view under a 20× objective are set forth in FIG. 2A. FIG. 2B shows nonviable Hoechst⁺ fragmented/condensed nuclei per field of view under a 20× objective. FIG. 2C shows pSer129⁺ object counts per Hoechst⁺ cell. FIG. 2D shows the area occupied by pSer129⁺ objects (area fraction) per Hoechst⁺ cell. FIG. 2E shows log₁₀ pSer129⁺ area fraction per Hoechst⁺ cell. Shown are the mean±S.D. or boxplots for n=4 independent cultures, each run in duplicate or triplicate. Log-transformed data in FIG. 2E are shown as boxplots (despite an n of 4) because log-transformed units were negative and the bars were therefore upside down. The area fraction under vehicle-treated conditions is not shown because some values were zero in the absence of fibril treatment and could not be log-transformed. *p≤0.0500,**p≤0.0100,***p≤0.0010, repeated measures (matching for each independent culture) two-way ANOVA/Bonferroni. Statistically significant effects of experimental Lewy body disease (i.e., fibril exposure) were observed in FIGS. 2C-D (p≤0.0500), but the statistical impact of preformed fibrils in multiplicity-adjusted pairwise comparisons was not added, to avoid figure crowding.

FIGS. 3A-C show that AG161-41 lowers α-synucleinopathy in primary hippocampal cultures. Primary rat hippocampal cultures were treated with AG161-41 and preformed α-synuclein fibrils for 10 days in vitro before immunostaining for hyperphosphorylated α-synuclein (pSer129). The Hoechst reagent stained all nuclei. Quantification of viable Hoechst⁺ cell counts per field of view under a 20× objective (FIG. 3A), FIG. 3B shows the area occupied by pSer129⁺ objects (area fraction) per Hoechst⁺ cell, and FIG. 3C shows average sizes of individual pSer129⁺ objects. Representative images of cells labeled with the Hoechst nuclear stain or immunolabeled for pSer129 α-synuclein were observed. Shown are the mean±S.D. of n=3 independent cultures, each run in duplicate or triplicate. *p≤0.0500,**p≤0.0100, two-way repeated measures (with matching for each independent culture) ANOVA/Bonferroni. Statistically significant effects of experimental Lewy body disease or fibril exposure were observed in panels B-D (p≤0.0500), but the statistical impact of preformed fibrils in multiplicity-adjusted pairwise comparisons was not added, to avoid figure crowding. Primary hippocampal cultures were treated with AG161-47 (8 μM) or AG161-41 (4 μM) for 10 days in vitro, along with preformed fibrils. Cultures were exposed to the nonionic surfactant and emulsifier Triton X-100 during fixation, for removal of soluble α-synuclein, and stained with the pan-nuclear marker Hoechst and antibodies against pSer129 α-synuclein. Control staining with omission of the primary antibody (Ab) was performed.

FIGS. 4A-G show that AG161-47 does not elicit robust changes in post-translational markers of microtubule stability. Primary hippocampal cultures were treated for 10 days in vitro with preformed fibrils, AG161-47, and nocodazole (or vehicle). Full-length representative immunoblots showing the loading control (Total Protein REVERT stain), pan α-tubulin, and detyrosinated, tyrosinated, or acetylated α-tubulin levels were observed. Quantification of the levels of detyrosinated α-tubulin (FIGS. 4A and 4B), tyrosinated α-tubulin (FIGS. 4C and 4D), and acetylated α-tubulin (FIGS. 4E and 4F) as a fraction of pan α-tubulin. Shown are boxplots or scatterplots of 4-6 independent cultures. For FIGS. 4A-G, n=6 in all groups, except 1 μg/mL fibril+8 μM AG161-47+5 nM nocodazole-treated group, where the n=4. *p≤0.0500, repeated measures (matching for each independent culture) two or three-way ANOVA/Bonferroni, or a two-tailed paired t test on the fibril-treated groups alone (please follow the arrows). a.u.=arbitrary units.

FIGS. 5A-5F show that AG161-47 uncouples the link between post-translational markers of microtubule stability status and Lewy-related pathologies. Correlations between microtubule stability markers as a function of pan α-tubulin or total protein levels, in the presence or absence of preformed α-synuclein fibrils (FIG. 5A), nocodazole (FIG. 5B), or AG161-47 (FIG. 5C). Note the lack of correlations between stability markers in FIGS. 5A-5C, unless fibrils, nocodazole, or AG161-47 were on board. In FIG. 5D, fibril exposure elicited an unexpected positive correlation between pSer129 α-synuclein and the fraction of α-tubulin that was detyrosinated (marker of microtubule stability), but AG161-47 reversed the association in FIG. 5E and also severed the correlation between pSer129 α-synuclein and the total mass of acetylated (stable) α-tubulin in FIG. 5F. Pearson or Spearman correlation analyses were completed and associations between two independent variables were deemed to be statistically significant only if the two-tailed p value was below 0.0500 and the r value was above 0.4000. Arrows point to the functional impact of fibrils, nocodazole, or AG161-47 on the associations between the indicated variables. a.u.=arbitrary units. AG161-47 uncouples the link between post-translational markers of microtubule stability status and Lewy-related pathologies.

FIGS. 6A and 6B show that AG161-47 lowers locomotor activity in an open arena, but does not affect α-synucleinopathic inclusion counts in an in vivo model of limbic-centered Lewy body disease. At 11 months of age, male CD-1 mice were stereotaxically infused in the bulbar subdivision of the AON (OB/AON) with 1 μL PBS or with 5 μg preformed α-synuclein fibrils in 1 μL PBS. Vehicle or AG161-47-containing tablets were introduced daily into the home cage from the day of the stereotaxic brain surgery until sacrifice at six weeks post-infusion. FIG. 6A shows weekly bodyweights, shown in grams and as % change in bodyweight over time. FIG. 6B shows a battery of behavior tests completed after six weeks of daily tablet consumption, including the open field test. Shown here are each animal's mean speed, number of rears, and distance traveled in the open field. Sagittal brain sections were immunostained for the neuronal nuclear marker NeuN and pSer129⁺ inclusions. The Hoechst reagent was also applied to stain all nuclei. The rostral and medial limits of the primary olfactory (piriform) cortex were analyzed. The n is 9 mice per group, and each mouse's datapoint is illustrated as a colored dot. FIG. 6A shows the mean±S.D. and FIG. 6B shows boxplots with interquartile ranges. For FIGS. 6B, an ordinary one-way ANOVA/Bonferroni was applied. Two-tailed p values are shown above each bracketed pairwise comparison.

FIG. 7A shows that AG161-47 lowers insoluble pathological α-synuclein in an in vivo model of limbic-centered Lewy body disease. At 11 months of age, male CD-1 mice were stereotaxically infused in the bulbar subdivision of the AON (OB/AON) with 1 μL PBS or with 5 g preformed α-synuclein fibrils in 1 μL PBS. Vehicle or AG161-47-containing tablets were introduced daily into the home cage from the day of the stereotaxic brain surgery until sacrifice at six weeks post-infusion. After six weeks, the piriform cortex and amygdala from each animal were pooled and fractionated by ultracentrifugation in Triton X-100. Immunoblotting for pSer129 α-synuclein and pan α-synuclein was then performed on the insoluble fraction. FIG. 7A shows from left to right quantification of the Total Protein REVERT stain as the loading control, insoluble pSer129 α-synuclein as a fraction of total protein, insoluble pan α-synuclein as a fraction of total protein, and insoluble pSer129 α-synuclein as a fraction of pan α-synuclein.

Representative full length immunoblots were observed (not shown). Note that the total protein stain is chemically removed prior to exposure to primary antibodies. FIG. 7B shows the correlations between the fraction of α-tubulin that was tyrosinated or the levels of acetylated α-tubulin and insoluble pan α-synuclein levels in fibril-infused mice. FIG. 7C shows the correlation of insoluble pSer129 α-synuclein levels with the fraction of α-tubulin that was acetylated in fibril-infused animals, in presence or absence of AG161-47 (5 mg/kg). FIG. 7D shows the correlations of detyrosinated α-tubulin levels with the fraction of insoluble α-synuclein that was hyperphosphorylated in fibril-infused mice, in presence or absence of AG161-47 (5 mg/kg). FIG. 7E shows the correlation of mean locomotor speed with insoluble pan α-synuclein in fibril-infused animals, in presence or absence of AG161-47 (5 mg/kg). The n is 9 mice per group, and each mouse's datapoint is illustrated as a colored dot. For FIGS. 7B-E, associations between the two independent variables were deemed to be statistically significant if the two-tailed p value of the correlation analysis was ≤0.0500 and the r value was above 0.4000. a.u.=arbitrary units.

FIG. 8 shows a synthesis route scheme for compounds AG182-126 and AG185-174 with the following reaction conditions: AG182-126: (a) i) N-methylbenzofuran-5-amine 2, n-BuOH, conc. HCl, 150° C., 4 hours, microwave reactor ii) 1N NaOH, reflux, 4 hours, 25%; AG185-174: (b) i) N-methyl-5,6,7,8-tetrahydronaphthalen-2-amine 3, i-PrOH, conc. HCl, reflux, 120 hours ii) 1N NaOH, reflux, 4 hours, 30%.

FIGS. 9A, 9B, 9C, 9D, 9E, 9F, and 9G show the impact of pyrimido[4,5-b]indoles on α-synucleinopathy in vitro. Primary hippocampal cultures were treated with preformed α-synuclein fibrils and the compounds indicated in FIG. 9A for 10 days in vitro before immunostaining for hyperphosphorylated α-synuclein (pSer129) as a marker of Lewy-like pathology, and the pan-neuronal marker βIII-tubulin as a viability marker (FIGS. 9B, 9C, 9D, 9E, 9F, and 9G).

Representative In-Cell Western analyses for pSer129 and βIII-tubulin were observed (not shown). FIGS. 9B, 9D, and 9E show fluorescence values for βIII-tubulin staining on the Odyssey Imager M. pSer129 immunoreactive signal was expressed as a function of βIII-tubulin levels in FIGS. 9C, 9F, and 9G. Shown are the mean±S.D. for n=4 independent cultures. *p≤0.0500, **p≤0.0100, two-way repeated measures ANOVA/Bonferroni (with matching for each independent culture). Main effects of fibril exposure (i.e., experimental Lewy body disease) are indicated above graphs. Statistically significant effects of experimental Lewy body disease or fibril exposure were observed (p≤0.0500), but the statistical impact of preformed fibrils in multiplicity-adjusted pairwise comparisons was not added to avoid figure crowding.

FIGS. 10A, 10B, 10C, and 10D show the impact of AG161-47 on tyrosinated, detyrosinated, and acetylated α-tubulin in vivo. At 11 months of age, male CD-1 mice were stereotaxically infused in the bulbar subdivision of the AON (OB/AON) with 1 μL PBS or with 5 g preformed α-synuclein fibrils in 1 μL PBS. Vehicle or AG161-47-containing tablets were introduced daily into the home cage from the day of the stereotaxic brain surgery until sacrifice at six weeks post-infusion. OB/AON tissues were harvested and subjected to SDS-PAGE/immunoblotting procedures for α-tubulin stabilization/destabilization markers. Representative full length immunoblots for indicated markers were observed (not shown). Please note that the loading control Total Protein REVERT stain is completely washed off prior to exposure to primary antibodies. FIG. 10A shows levels of acetylated α-tubulin as a fraction of pan α-tubulin and total protein. FIG. 10B shows levels of detyrosinated α-tubulin as a fraction of pan α-tubulin and total protein. FIG. 10C shows levels of tyrosinated α-tubulin as a fraction of pan α-tubulin and total protein. For FIGS. 10A-10C, an ordinary one-way ANOVA/Bonferroni was applied but no statistically significant changes were identified.

FIGS. 11A, 11B, 11C, and 11D show the impact of biological sex on tyrosinated α-tubulin in the human amygdala. Amygdala samples harvested postmortem from individuals diagnosed with or without Lewy body disorders were subjected to SDS-PAGE and immunoblotting for detyrosinated, acetylated, or tyrosinated (FIG. 11A) α-tubulin. The REVERT Total Protein Stain was employed as the loading control. Only the tyrosinated α-tubulin blots are shown, as the other antibodies were subpar on amygdala tissues. As no statistically significant effect of Lewy body disease on tyrosinated α-tubulin was observed, all data were collapsed in FIGS. 11B-11D to focus on the effect of sex with higher power. Data in FIG. 11A were analyzed by two-way ANOVA/Bonferroni, and the remaining panels show **p≤0.0100 by a two-tailed, unpaired t-test.

Animal model results: As AG161-47 remains the most efficacious of all the tricyclics we have tested to date, we evaluated its therapeutic potential in our established in vivo model of limbic Lewy body disease. For further descriptions of the animal model, please consult our published work. (Bhatia et al., 2021; Bhatia et al., 2023; Mason et al., 2016; Mason et al., 2019; Miner et al., 2022; Nouraei et al., 2018) Our work was significantly delayed, as we had to develop a new oral delivery platform for compounds, to avoid AG161-47 dissolution in water or any application of heat. We compressed AG161-47 with nutrients into a palatable tablet with an ideal consistency for rapid rodent consumption without loss of material to rodent food-grinding behaviors. Our tablets (described herein) completely avoid the daily stress of intraperitoneal or subcutaneous injections with needles, or oral gavage methods, where food is injected directly into the animal's stomach via the throat.

Using the tablet-based drug delivery method, we completed stereotaxic brain surgeries, daily tablet administration, behavior tests, and tissue harvesting in our in vivo model of limbic Lewy body disease. Thus, male CD-1 mice were infused in the olfactory bulb with one microliter of phosphate-buffered saline (PBS) or 5 micrograms of preformed α-synuclein fibrils in one microliter of PBS, to induce an experimental model of early-stage, limbic-centered Lewy body disease at eight months of age, as previously described. (Bhatia et al., 2021; Bhatia et al., 2023; Mason et al., 2016; Mason et al., 2019; Miner et al., 2022; Nouraei et al., 2018)

Animals infused with fibrils were then exposed to one tablet with 0 (placebo), 5, or 10 mg/kg AG161-47 (n=9 mice per group) every day for the next six weeks. The majority of mice consumed the entire tablet per day over the course of six sequential weeks of testing. Thus, 26 out of 36 mice consumed all tablets every day following day 1 of exposure, over the course of six sequential weeks, 72% of mice consumed 100% of these tablets after day 1, and 89% of mice consumed 80% or more of tablets after day 1. No loss of bodyweight was elicited by exposure to AG161-47 compared to the placebo group. Exposure to AG161-47 reduced the slightly overactive phenotype that was induced at early stages of disease (six weeks after fibril infusions. No other behavior deficits were noted in this model, likely due to the early survival period (six weeks after fibril infusions).

To improve the precision of our work, we recently optimized a method of detergent-based fractionation of insoluble vs. soluble α-synuclein using ultracentrifugation. (Bhatia et al., 2023) In Lewy body disorders as well as the fibril model, α-synuclein becomes insoluble in the nonionic detergent Triton X-100 as well as pathologically phosphorylated at the serine 129 (pSer129) residue, a post-translational modification that is visualized with use of a specific monoclonal antibody EP1536Y, (Delic et al., 2018; Volpicelli-Daley, Luk, et al., 2014) which we also used here. We have applied this validated biochemical technique to extracts of the amygdala and piriform cortex, two brain regions that form dense pSer129 α-synuclein⁺ inclusions in our animal model. (Bhatia et al., 2021; Bhatia et al., 2023; Mason et al., 2016; Mason et al., 2019; Miner et al., 2022; Nouraei et al., 2018) We observed that the 5 mg/kg dose of AG161-47 reduced the pathologically phosphorylated fraction of insoluble α-synuclein. We believe that the 10 mg/kg dose was too high to exert beneficial effects on Lewy-related pathology. We are now continuing to mine these animal tissues and behavior data and expect to submit a paper on the in vivo effects of AG161-47. Future studies will examine 1.25 and 2.5 mg/kg doses of AG161-47 for longer survival periods.

FIGS. 6A, 6B, and 7A show the impact of AG161-47 in an animal model of Lewy Body disease. At 8 months of age, genetically outbred male CD-1 mice were infused in the olfactory bulb with sonicated, preformed α-synuclein fibrils (5 μg in 1 μL) or an equivalent volume of phosphate buffered saline (PBS) as the vehicle control. Starting on the next day, all animals were treated with a palatable tablet, in which we packed 0, 5, or 10 mg/kg bodyweight of AG161-47 (n=9 per group) with cellulose and simple and complex carbohydrates, fats, and proteins. A heatmap of tablet ingestion over the course of six weeks after fibril infusion surgeries was observed (not shown). FIG. 6A shows bodyweights were assessed for the duration of the study and expressed in grams or as % change from presurgical baseline values. FIG. 6B shows locomotor behaviors were measured in an open field in the days before sacrifice (six weeks post-surgery), with use of ANY-Maze software by a blinded observer. FIG. 7A shows biochemical extraction of nonionic detergent-insoluble and pathologically phosphorylated (pSer129) α-synuclein from amygdala and piriform cortical tissues of all mice via ultracentrifugation of Triton X-100 insoluble fraction at 100,000×g and resuspension in sodium dodecyl sulfate, followed by SDS-PAGE/infrared immunoblotting on an Odyssey M imager. P values are shown per one or two-way ANOVA analyses and Bonferroni post hoc testing. In conclusion, we have observed that application of AG161-47 lowers the pathological form of α-synuclein in primary neurons cultured in vitro as well as in an animal model of Lewy body disease. AG161-47 is also known as Compound 11 herein and as Compound 3 in our published work on its dual-acting effects on cellular microtubules. (Rohena et al., 2016) Thus, the collective results to date support the view that the microtubule modulator AG161-47 diminishes Lewy-related pathologies in primary rat neurons in vitro and in mice in vivo.

TABLE 1
Primary Antibodies

| Antigen | Host | Manufacturer | Cat. No. | Lot. No. | Dilution |
|---|---|---|---|---|---|
| Pan α-tubulin | Rabbit | Sigma (Clone RM113) | SAB5600206 | S0602286, | 1:1000 |
| | | | | V0405082 | |
| Pan α-tubulin | Mouse | Sigma (Clone B-5-1-2) | T5168 | 84283 | 1:100,000 |
| Tyrosinated α-tubulin | Mouse | Sigma (Clone TUB-1A2) | T9028 | 029M4897V | 1:800 |
| Detyrosinated α-tubulin | Rabbit | Millipore | AB3201 | 3584332, 3536025 | 1:500 |
| Acetylated α-tubulin | Mouse | Sigma (Clone 6-11B-1) | T7451 | 078M4842V | 1:10,000 |
| pSer129 α-synuclein | Rabbit | Abcam (Clone EP1536Y) | AB51253 | GR3232346-14 | 1:1000 |
| | | | | GR3317474-4 | |
| | | | | GR3191908-7 | |
| Pan α-Synuclein | Mouse | BD Biosciences (Clone 42) | AB610786 | 0314065 | 1:1000 |
| KIF5B | Rabbit | Cell Signaling Technologies | 18148S | 1 | 1:500 |
| Dynein | Mouse | Invitrogen (Clone 74.1) | MA1-070 | VI307791 | 1:500 |
| MAP2 | Mouse | Sigma (Clone HM-2) | M9942 | 018M4785V | 1:750 |
| β-III tubulin | Rabbit | Cell Signaling Technologies | 5568S | 6 | 1:300 |
| | | (Clone D71G9) | | | |
| β-III tubulin | Chicken | Aves Labs | TUJ | TUJ89947984 | 1:2000 |
| NeuN | Guinea Pig | Millipore | ABN90 | 2610504 | 1:6000 |

TABLE 2
Secondary Antibodies

| Antigen | Tag | Host | Manufacturer | Cat. No. | Lot No. | Dilution |
|---|---|---|---|---|---|---|
| Rabbit IgG | Alexa | Donkey | Jackson | 711-655-152 | 132235 | (WB) 1:15,000- |
| | Fluor 790 | | ImmunoResearch | | | 30,000 |
| | | | Laboratories Inc. | | | (ICC) 1:700 |
| Rabbit IgG | Alexa | Donkey | Jackson | 711-625-152 | 135980 | (WB) 1:15,000- |
| | Fluor 680 | | ImmunoResearch | | | 30,000 |
| | | | Laboratories Inc. | | | (ICC) 1:700 |
| Rabbit IgG | Cy3 | Donkey | Jackson | 711-165-152 | 154879 | (WB) 1:15,000- |
| | | | ImmunoResearch | | | 30,000 |
| | | | Laboratories Inc. | | | (ICC) 1:700 |
| Mouse IgG | Alexa | Donkey | Jackson | 715-655-151 | 133569 | (WB) 1:15,000- |
| | Fluor 790 | | ImmunoResearch | | | 30,000 |
| | | | Laboratories Inc. | | | (ICC) 1:700 |
| Mouse IgG | Alexa | Donkey | Jackson | 715-625-151 | 127891 | (WB) 1:15,000- |
| | Fluor 680 | | ImmunoResearch | | | 30,000 |
| | | | Laboratories Inc. | | | (ICC) 1:700 |
| Mouse IgG | Alexa | Donkey | Jackson | 715-545-150 | 130996 | (WB) 1:15,000- |
| | Fluor 488 | | ImmunoResearch | | | 30,000 |
| | | | Laboratories Inc. | | | (ICC) 1:700 |
| Chicken IgY | Alexa | Donkey | Jackson | 703-605-155 | 138591 | (WB) 1:15,000- |
| | Fluor 647 | | ImmunoResearch | | | 30,000 |
| | | | Laboratories Inc. | | | (ICC) 1:700 |
| Chicken IgY | Alexa | Donkey | Jackson | 703-625-155 | 136950 | (WB) 1:15,000- |
| | Fluor 680 | | ImmunoResearch | | | 30,000 |
| | | | Laboratories Inc. | | | (ICC) 1:700 |
| Guinea Pig IgG | Alexa | Donkey | Jackson | 706-655-148 | 154634 | (IHC) 1:500 |
| | Fluor 790 | | ImmunoResearch | | | |
| | | | Laboratories Inc. | | | |
| Rabbit IgG | Horseradish | Donkey | Jackson | 711-035-152 | 157399 | (IHC) 1:160 |
| | Peroxidase | | ImmunoResearch | | | |
| | | | Laboratories Inc. | | | |

TABLE 3
Doses per Unit Bodyweight per Animal mg/kg bodywgt.

| | Bodyweight | Target Dose | Milligram | Final Dose |
|---|---|---|---|---|
| | (kg) | (mg/kg) | dose | per tablet |
| PBS Placebo | 0.043 | 0 | 0.00 | 0.00 |
| Tablet | 0.046 | 0 | 0.00 | 0.00 |
| | 0.045 | 0 | 0.00 | 0.00 |
| | 0.045 | 0 | 0.00 | 0.00 |
| | 0.049 | 0 | 0.00 | 0.00 |
| | 0.042 | 0 | 0.00 | 0.00 |
| | 0.053 | 0 | 0.00 | 0.00 |
| | 0.050 | 0 | 0.00 | 0.00 |
| | 0.043 | 0 | 0.00 | 0.00 |
| Fibril Placebo | 0.040 | 0 | 0.00 | 0.00 |
| Tablet | 0.053 | 0 | 0.00 | 0.00 |
| | 0.048 | 0 | 0.00 | 0.00 |
| | 0.047 | 0 | 0.00 | 0.00 |
| | 0.058 | 0 | 0.00 | 0.00 |
| | 0.040 | 0 | 0.00 | 0.00 |
| | 0.049 | 0 | 0.00 | 0.00 |
| | 0.056 | 0 | 0.00 | 0.00 |
| | 0.051 | 0 | 0.00 | 0.00 |
| Fibril Low-Dose | 0.041 | 5 | 0.25 | 6.10 |
| Tablet | 0.051 | 5 | 0.25 | 4.90 |
| | 0.055 | 5 | 0.25 | 4.55 |
| | 0.046 | 5 | 0.25 | 5.43 |
| | 0.046 | 5 | 0.25 | 5.43 |
| | 0.047 | 5 | 0.25 | 5.32 |
| | 0.045 | 5 | 0.25 | 5.56 |
| | 0.044 | 5 | 0.25 | 5.68 |
| | 0.049 | 5 | 0.25 | 5.10 |
| Fibril High-Dose | 0.045 | 10 | 0.50 | 11.11 |
| tablet | 0.044 | 10 | 0.50 | 11.36 |
| | 0.047 | 10 | 0.50 | 10.64 |
| | 0.058 | 10 | 0.50 | 8.62 |
| | 0.050 | 10 | 0.50 | 10.00 |
| | 0.045 | 10 | 0.50 | 11.11 |
| | 0.045 | 10 | 0.50 | 11.11 |
| | 0.055 | 10 | 0.50 | 9.09 |
| | 0.048 | 10 | 0.50 | 10.42 |

TABLE 4
Demographics for postmortem samples from UCLA NIH NeuroBioBank

| | | | | | Neuropathological | Clinical |
|---|---|---|---|---|---|---|
| Pt.Id. | Age | Sex | LBD | PMI | Diagnosis | Diagnosis |
| 3298 | 79 | M | Control | 20.0 | Recent cerebrum hypoxia | CA, prostate, Fetal failure (acute), |
| | | | | | | Diabetes Type 1 |
| 3336 | 77 | M | Control | 15.5 | Cerebrum hypoxia | Heart attack, CA, prostate |
| | | | | | (intermediate) | |
| 3451 | 82 | M | Control | 14.0 | Minor aging changes | Congestive heart failure |
| 3345 | 82 | F | Control | 12.0 | Recent cerebrum hypoxia | CA, colon |
| 3795 | 72 | F | Control | 14.0 | Atherosclerosis (moderate), | CA, ovarian; CA, peri [symbol] al |
| | | | | | No evidence of metastatic | |
| | | | | | carcinoma | |
| 3 [symbol] 24 | 79 | F | Control | 52.0 | Multiple sclerosis (clinical) | Multiple sclerosis, MRSA (Methicillin |
| | | | | | | resistant staphylococcus aureus), |
| | | | | | | Seizures, Chronic obstructive pulmonary |
| | | | | | | disease, C [symbol] , Parapl [symbol] , Hy [symbol] , |
| | | | | | | Ga [symbol] reflux disorder |
| 4002 | 82 | M | Diseased | 19.5 | PD, Atherosclerosis | PD, Lewy Body disease, Depression, Dementia, |
| | | | | | | Senile, Atherosclerosis, Sleep disorder, |
| | | | | | | Hypothyroidism |
| 4220 | 68 | M | Diseased | 13.8 | AD (probable), | AD, LBD, Lewy Body disease (probable), |
| | | | | | Lewy body disease (cortical) | Hypertension, Oste [symbol] |
| 4365 | 71 | M | Diseased | 20.3 | LBD | LBD, Depression, Hypertension, Diabetes |
| | | | | | | Type II, Sleep disorder, Alcohol abuse |
| 41 [symbol] 0 | 82 | F | Diseased | 12.3 | AD (probable), atherosclerosis | AD, Organic brain syndrome (OBS), Dementia, |
| | | | | | (moderate) | Depression, Lewy Body disease, A [symbol] , |
| | | | | | | T [symbol] Attack ( [symbol] ), Renal failure |
| | | | | | | (acute), Congestive heart failure, CA [symbol] |
| 4155 | 79 | F | Diseased | 17.7 | AD (definite), LBD | AD, Cognitive impairment, Depression, Dementia, |
| | | | | | | Chronic obstructive pulmonary disease |
| 4136 | 75 | F | Diseased | 15.2 | AD (probable), LBD | LBD, PD, Vascular dementia (possible), AD, |
| | | | | | | Hypothyroidism, Urinary tract infection, |
| | | | | | | Depression, Psychosis, Obsessive |
| | | | | | | compulsive disorder |

[symbol]

indicates data missing or illegible when filed

TABLE 5
Demographics for postmortem samples from U. Miami NIH NeuroBioBank

| | | | | | Neuropathological | | | |
|---|---|---|---|---|---|---|---|---|
| Subject | Age | Sex | LBD | PMI | Diagnosis | Race | Ethnicity | Hemisphere(s) |
| Hct15HAUA006 | 65 | M | Control | 21.8 | Unaffected | White | Hispanic | Right |
| | | | | | Control | | or Latino | |
| Hct15HBQA005 | 66 | M | Control | 6.83 | Unaffected | White | Hispanic | Right |
| | | | | | Control | | or Latino | |
| Hct15HBCA003 | 83 | M | Control | 25 | Unaffected | White | Hispanic | Both |
| | | | | | Control | | or Latino | |
| HctZZTA003 | 84 | M | Control | 15.5 | Unaffected | White | Not Hispanic | Both |
| | | | | | Control | | or Latino | |
| HBKVA003 | 74 | M | Diseased | 8.98 | Dementia with | White | Not Hispanic | Both |
| | | | | | Lewy Bodies | | or Latino | |
| BEB18025A002 | 79 | M | Diseased | 23.66 | Dementia with | White | Not Hispanic | Right |
| | | | | | Lewy Bodies | | or Latino | |
| HBKZA003 | 80 | M | Diseased | 28 | Dementia with | White | Not Hispanic | Left |
| | | | | | Lewy Bodies | | or Latino | |
| HBKTA003 | 81 | M | Diseased | 29 | Dementia with | White | Not Hispanic | Left |
| | | | | | Lewy Bodies | | or Latino | |
| HctZZHA003 | 65 | F | Control | 19.43 | Unaffected | White | Not Hispanic | Both |
| | | | | | Control | | or Latino | |
| HctZZCA003 | 82 | F | Control | 14.2 | Unaffected | White | Not Hispanic | Both |
| | | | | | Control | | or Latino | |
| HctZZVA003 | 86 | F | Control | 25.11 | Unaffected | White | Not Hispanic | Both |
| | | | | | Control | | or Latino | |
| HBJRA003 | 73 | F | Diseased | 7.71 | Dementia with | White | Hispanic | Left |
| | | | | | Lewy Bodies | | or Latino | |
| HBNMA003 | 79 | F | Diseased | 20.95 | Dementia with | White | Not Hispanic | Both |
| | | | | | Lewy Bodies | | or Latino | |
| HBNFA003 | 83 | F | Diseased | 25.08 | Dementia with | White | Not Hispanic | Left |
| | | | | | Lewy Bodies | | or Latino | |

Table 6 provides kinase inhibition and inhibitor validation data for each kinase tested. The representative IC₅₀ value of the Std mined at the ATP bin nearest to the ATP Km app. AGN5 is also identified as AG 161-41 and AGN11 is also identified as AG 161-47.

TABLE 6

| | | | | ATP (μM) | | IC₅₀ (nM) |
|---|---|---|---|---|---|---|
| | IC₅₀ (nM) of | | Std | Km | | of Std |
| Kinase | AGN5 | AGN11 | inhibitor | app | Bin | inhibitor |
| ABL1 | 26.1 | 7.96 | Tyrphostin | 12 | 10 | 326 |
| | | | AG1478 | | | |
| CSNK1A1 | 498 | 30.5 | TBB | 2 | 5 | 12600 |
| (CK1 alpha 1) | | | | | | |
| CSNK2A2 | >1110 | >1110 | TBB | 46 | 50 | 2170 |
| (CK2 alpha 2) | | | | | | |
| DYRK1A | 249 | 8.15 | Staurosporine | 99.7 | 100 | 25.6 |
| FYN | 169 | 888 | Tyrphostin | 85 | 75 | 5300 |
| | | | AG1478 | | | |
| GRK5 | >370 | >1110 | Staurosporine | 3 | 5 | 144 |
| GRK6 | 64.3 | 614 | Staurosporine | 11.7 | 10 | 59 |
| GSK3β | 3.12 | 8.12 | Staurosporine | 7 | 10 | 10.8 |
| LARK2 | 20 | 7.03 | Staurosporine | 70 | 75 | 1.64 |
| PLK2 | 16.7 | >370 | Staurosporine | 29.6 | 25 | 367 |

Table 7 shows the data of a pharmacokinetic study.

TABLE 7

| Study | | | | AGN5 | AGN11 |
|---|---|---|---|---|---|
| In silico | MW | | | 337.31 | 323.78 |
| Solubility | pH 2.0 (μM) | | | 1.75 | 0.71 |
| Protein Binding | Human Plasma | | % Bound | 99.78 | 99.60 |
| | Mouse Plasma | | | >99.98* | 99.78 |
| Brain Tissue Binding | Mouse | | | 99.96 | 99.92 |
| Metabolic Stability | Human | T_{1/2} | min | 38.88 | 38.10 |
| | | Clint | mL/min/Kg | 44.71 | 45.63 |
| | Mouse | T_{1/2} | min | 10.08 | 10.07 |
| | | Clint | mL/min/Kg | 601.58 | 602.12 |
| Permeability | | Papp (A-B) | (10−6, cm/s) | 1.74 | 4.97 |
| | MDC-MDR1 | Papp (B-A) | (10−6, cm/s) | 2.98 | 4.16 |
| | | Efflux Ratio | | 1.71 | 0.84 |
| CD-1 Mice PK | IV | CI_{obs} | mL/min/Kg | 49.5 | 120 |
| IV1 mpk | | T_{1/2} | h | 1.55 | 0.324 |
| PO 1 mpk | | C₀ | ng/mL | 811 | 529 |
| | | Vss_obs | L/kg | 3.24 | 2.41 |
| | PO | T½ | h | 0.78 | 0.60 |
| | | Tmax | h | 0.500 | 0.417 |

| | | | | | |
|---|---|---|---|---|---|
| Cmax | ng/mL | 14.7 | 44 nM | 9.7 | 30 nM |

| | | | |
|---|---|---|---|
| AUClast | h*ng/mL | 19.2 | 11.0 |
| F | % | 6.35 | 8.38 |

| | | | |
|---|---|---|---|
| Brain/Plasma Ratio (Kp brain) | Mean | 5.99 | 4.5 |
| 2 hr Kpuu = [Cu, brain/Cu, plasma] | | 6.0 | 4.4 |

### Synthesis Scheme for Compounds AGN5 and AGN11:

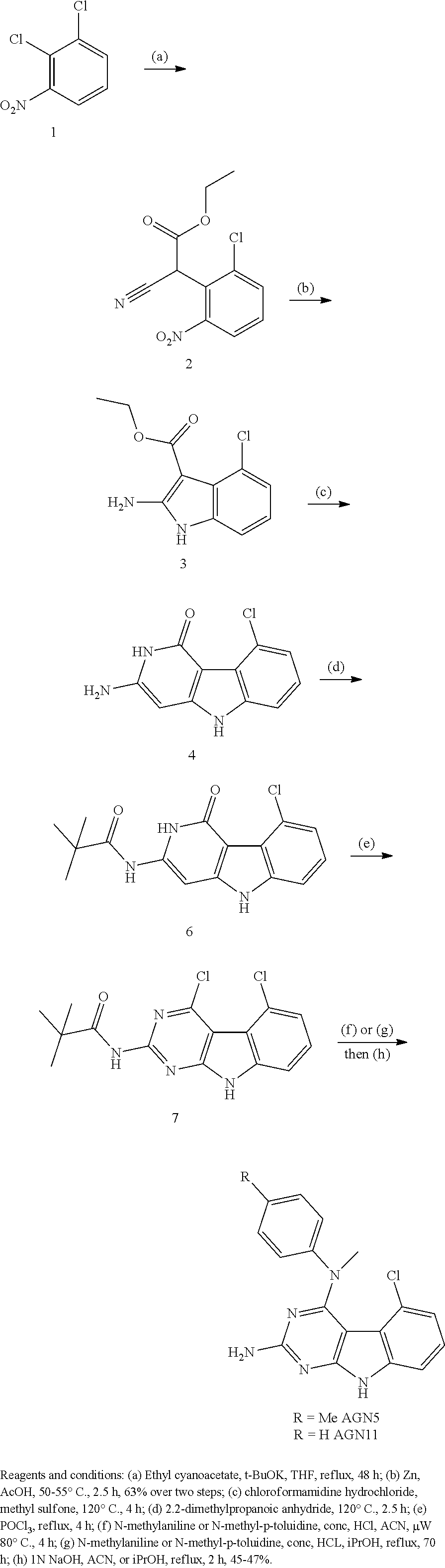

### NON-NUMBERED REFERENCES OF THE SPECIFICATION

Akhmanova, A., & Kapitein, L. C. (2022). Mechanisms of microtubule organization in differentiated animal cells. Nat Rev Mol Cell Biol, 23(8), 541-558. https://doi.org/10.1038/s41580-022-00473-y
Aldinger, K. A., Sokoloff, G., Rosenberg, D. M., Palmer, A. A., & Millen, K. J. (2009). Genetic variation and population substructure in outbred CD-1 mice: implications for genome-wide association studies. PLoS One, 4(3), e4729. https://doi.org/10.1371/journal.pone.0004729
Anderson, E. N., Hirpa, D., Zheng, K. H., Banerjee, R., & Gunawardena, S. (2019). The Non-amyloidal Component Region of alpha-Synuclein Is Important for alpha-Synuclein Transport Within Axons. Front Cell Neurosci, 13, 540. https://doi.org/10.3389/fncel.2019.00540
Anderson, J. P., Walker, D. E., Goldstein, J. M., de Laat, R., Banducci, K., Caccavello, R. J., Barbour, R., Huang, J., Kling, K., Lee, M., Diep, L., Keim, P. S., Shen, X., Chataway, T., Schlossmacher, M. G., Seubert, P., Schenk, D., Sinha, S., Gai, W. P., & Chilcote, T. J. (2006). Phosphorylation of Ser-129 is the dominant pathological modification of alpha-synuclein in familial and sporadic Lewy body disease. J Biol Chem, 281(40), 29739-29752. https://doi.org/10.1074/jbc.M600933200
Arce, C. A., Rodriguez, J. A., Barra, H. S., & Caputo, R. (1975). Incorporation of L-tyrosine, L-phenylalanine and L-3,4-dihydroxyphenylalanine as single units into rat brain tubulin. Eur J Biochem, 59(1), 145-149. https://doi.org/10.1111/j.1432-1033.1975.tb02435.x
Baas, P. W., & Black, M. M. (1990). Individual microtubules in the axon consist of domains that differ in both composition and stability. J Cell Biol, 111(2), 495-509. https://doi.org/10.1083/jcb.111.2.495
Baas, P. W., Rao, A. N., Matamoros, A. J., & Leo, L. (2016). Stability properties of neuronal microtubules. Cytoskeleton (Hoboken), 73(9), 442-460. https://doi.org/10.1002/cm.21286
Bates, D., & Eastman, A. (2017). Microtubule destabilising agents: far more than just antimitotic anticancer drugs. Br J Clin Pharmacol, 83(2), 255-268. https://doi.org/10.1111/bcp.13126
Bendiske, J., Caba, E., Brown, Q. B., & Bahr, B. A. (2002). Intracellular deposition, microtubule destabilization, and transport failure: an “early” pathogenic cascade leading to synaptic decline. J Neuropathol Exp Neurol, 61(7), 640-650. https://www.ncbi.nlm.nih.gov/pubmed/12125743
Bhatia, T. N., Clark, R. N., Needham, P. G., Miner, K. M., Jamenis, A. S., Eckhoff, E. A., Abraham, N., Hu, X., Wipf, P., Luk, K. C., Brodsky, J. L., & Leak, R. K. (2021). Heat Shock Protein 70 as a Sex-Skewed Regulator of alpha-Synucleinopathy. Neurotherapeutics, 18(4), 2541-2564. https://doi.org/10.1007/s13311-021-01114-6
Bhatia, T. N., Jamenis, A. S., Abbas, M., Clark, R. N., Miner, K. M., Chandwani, M. N., Kim, R. E., Hilinski, W., O'Donnell, L. A., Luk, K. C., Shi, Y., Hu, X., Chen, J., Brodsky, J. L., & Leak, R. K. (2023). A 14-day pulse of PLX5622 modifies alpha-synucleinopathy in preformed fibril-infused aged mice of both sexes. Neurobiol Dis, 106196. https://doi.org/10.1016/j.nbd.2023.106196
Braak, H., Bohl, J. R., Muller, C. M., Rub, U., de Vos, R. A., & Del Tredici, K. (2006). Stanley Fahn Lecture 2005: The staging procedure for the inclusion body pathology associated with sporadic Parkinson's disease reconsidered. Mov Disord, 21(12), 2042-2051. https://doi.org/10.1002/mds.21065
Brouhard, G. J., & Rice, L. M. (2018). Microtubule dynamics: an interplay of biochemistry and mechanics. Nat Rev Mol Cell Biol, 19(7), 451-463. https://doi.org/10.1038/s41580-018-0009-y
Brunden, K. R., Lee, V. M., Smith, A. B., 3rd, Trojanowski, J. Q., & Ballatore, C. (2017). Altered microtubule dynamics in neurodegenerative disease: Therapeutic potential of microtubule-stabilizing drugs. Neurobiol Dis, 105, 328-335. https://doi.org/10.1016/j.nbd.2016.12.021
Canty, J. T., Tan, R., Kusakci, E., Fernandes, J., & Yildiz, A. (2021). Structure and Mechanics of Dynein Motors. Annu Rev Biophys, 50, 549-574. https://doi.org/10.1146/annurev-biophys-111020-101511
Carnwath, T., Mohammed, R., & Tsiang, D. (2018). The direct and indirect effects of alpha-synuclein on microtubule stability in the pathogenesis of Parkinson's disease. Neuropsychiatr Dis Treat, 14, 1685-1695. https://doi.org/10.2147/NDT.S166322
Cartelli, D., Aliverti, A., Barbiroli, A., Santambrogio, C., Ragg, E. M., Casagrande, F. V., Cantele, F., Beltramone, S., Marangon, J., De Gregorio, C., Pandini, V., Emanuele, M., Chieregatti, E., Pieraccini, S., Holmqvist, S., Bubacco, L., Roybon, L., Pezzoli, G., Grandori, R., . . . Cappelletti, G. (2016). alpha-Synuclein is a Novel Microtubule Dynamase. Sci Rep, 6, 33289. https://doi.org/10.1038/srep33289
Cartelli, D., & Cappelletti, G. (2017). Microtubule Destabilization Paves the Way to Parkinson's Disease. Mol Neurobiol, 54(9), 6762-6774. https://doi.org/10.1007/s12035-016-0188-5
Cartelli, D., Casagrande, F., Busceti, C. L., Bucci, D., Molinaro, G., Traficante, A., Passarella, D., Giavini, E., Pezzoli, G., Battaglia, G., & Cappelletti, G. (2013). Microtubule alterations occur early in experimental parkinsonism and the microtubule stabilizer epothilone D is neuroprotective. Sci Rep, 3, 1837. https://doi.org/10.1038/srep01837
Cartelli, D., Goldwurm, S., Casagrande, F., Pezzoli, G., & Cappelletti, G. (2012). Microtubule destabilization is shared by genetic and idiopathic Parkinson's disease patient fibroblasts. PLoS One, 7(5), e37467. https://doi.org/10.1371/journal.pone.0037467
Chauvin, S., & Sobel, A. (2015). Neuronal stathmins: a family of phosphoproteins cooperating for neuronal development, plasticity and regeneration. Prog Neurobiol, 126, 1-18. https://doi.org/10.1016/j.pneurobio.2014.09.002
Choi, W. S., Palmiter, R. D., & Xia, Z. (2011). Loss of mitochondrial complex I activity potentiates dopamine neuron death induced by microtubule dysfunction in a Parkinson's disease model. J Cell Biol, 192(5), 873-882. https://doi.org/10.1083/jcb.201009132
Chu, Y., Morfini, G. A., Langhamer, L. B., He, Y., Brady, S. T., & Kordower, J. H. (2012). Alterations in axonal transport motor proteins in sporadic and experimental Parkinson's disease. Brain, 135(Pt 7), 2058-2073. https://doi.org/10.1093/brain/aws133
Cooper, A. A., Gitler, A. D., Cashikar, A., Haynes, C. M., Hill, K. J., Bhullar, B., Liu, K., Xu, K., Strathearn, K. E., Liu, F., Cao, S., Caldwell, K. A., Caldwell, G. A., Marsischky, G., Kolodner, R. D., Labaer, J., Rochet, J. C., Bonini, N. M., & Lindquist, S. (2006). Alpha-synuclein blocks E R-Golgi traffic and Rabl rescues neuron loss in Parkinson's models. Science, 313(5785), 324-328. https://doi.org/10.1126/science.1129462
Del Tredici, K., & Braak, H. (2016). Review: Sporadic Parkinson's disease: development and distribution of alpha-synuclein pathology. Neuropathol Appl Neurobiol, 42(1), 33-50. https://doi.org/10.1111/nan.12298
Delic, V., Chandra, S., Abdelmotilib, H., Maltbie, T., Wang, S., Kem, D., Scott, H. J., Underwood, R. N., Liu, Z., Volpicelli-Daley, L. A., & West, A. B. (2018). Sensitivity and Specificity of Phospho-Ser129 alpha-Synuclein Monoclonal Antibodies. J Comp Neurol, 526(12), 1978-1990. https://doi.org/10.1002/cne.24468
Dogterom, M., & Koenderink, G. H. (2019). Actin-microtubule crosstalk in cell biology. Nat Rev Mol Cell Biol, 20(1), 38-54. https://doi.org/10.1038/s41580-018-0067-1
Eidet, J. R., Pasovic, L., Maria, R., Jackson, C. J., & Utheim, T. P. (2014). Objective assessment of changes in nuclear morphology and cell distribution following induction of apoptosis. Diagn Pathol, 9, 92. https://doi.org/10.1186/1746-1596-9-92
Etienne-Manneville, S. (2010). From signaling pathways to microtubule dynamics: the key players. Curr Opin Cell Biol, 22(1), 104-111. https://doi.org/10.1016/j.ceb.2009.11.008
Ferreira, L. T., Figueiredo, A. C., Orr, B., Lopes, D., & Maiato, H. (2018). Dissecting the role of the tubulin code in mitosis. Methods Cell Biol, 144, 33-74. https://doi.org/10.1016/bs.mcb.2018.03.040
Franklin, K. B. J., & Paxinos, G. (2013). Paxinos and Franklin's The mouse brain in stereotaxic coordinates (Fourth edition. ed.). Academic Press, an imprint of Elsevier.
Freundt, E. C., Maynard, N., Clancy, E. K., Roy, S., Bousset, L., Sourigues, Y., Covert, M., Melki, R., Kirkegaard, K., & Brahic, M. (2012). Neuron-to-neuron transmission of alpha-synuclein fibrils through axonal transport. Ann Neurol, 72(4), 517-524. https://doi.org/10.1002/ana.23747
Gangjee, A., Zaware, N., Raghavan, S., Ihnat, M., Shenoy, S., & Kisliuk, R. L. (2010). Single agents with designed combination chemotherapy potential: synthesis and evaluation of substituted pyrimido[4,5-b]indoles as receptor tyrosine kinase and thymidylate synthase inhibitors and as antitumor agents. J Med Chem, 53(4), 1563-1578. https://doi.org/10.1021/jm9011142
Gitler, A. D., Bevis, B. J., Shorter, J., Strathearn, K. E., Hamamichi, S., Su, L. J., Caldwell, K. A., Caldwell, G. A., Rochet, J. C., McCaffery, J. M., Barlowe, C., & Lindquist, S. (2008). The Parkinson's disease protein alpha-synuclein disrupts cellular Rab homeostasis. Proc Natl Acad Sci USA, 105(1), 145-150. https://doi.org/10.1073/pnas.0710685105
Goedert, M., Spillantini, M. G., Del Tredici, K., & Braak, H. (2013). 100 years of Lewy pathology. Nat Rev Neurol, 9(1), 13-24. https://doi.org/10.1038/nrneurol.2012.242
Gudimchuk, N. B., & McIntosh, J. R. (2021). Regulation of microtubule dynamics, mechanics and function through the growing tip. Nat Rev Mol Cell Biol, 22(12), 777-795. https://doi.org/10.1038/s41580-021-00399-x
Gundersen, G. G., Kalnoski, M. H., & Bulinski, J. C. (1984). Distinct populations of microtubules: tyrosinated and nontyrosinated alpha tubulin are distributed differently in vivo. Cell, 38(3), 779-789. https://doi.org/10.1016/0092-8674(84)90273-3
Hasegawa, M., Fujiwara, H., Nonaka, T., Wakabayashi, K., Takahashi, H., Lee, V. M., Trojanowski, J. Q., Mann, D., & Iwatsubo, T. (2002). Phosphorylated alpha-synuclein is ubiquitinated in alpha-synucleinopathy lesions. J Biol Chem, 277(50), 49071-49076. https://doi.org/10.1074/jbc.M208046200
Hawkes, C. H., Del Tredici, K., & Braak, H. (2010). A timeline for Parkinson's disease. Parkinsonism Relat Disord, 16(2), 79-84. https://doi.org/S1353-8020(09)00217-X [pii]10.1016/j.parkreldis.2009.08.007
Hirokawa, N., Noda, Y., Tanaka, Y., & Niwa, S. (2009). Kinesin superfamily motor proteins and intracellular transport. Nat Rev Mol Cell Biol, 10(10), 682-696. https://doi.org/10.1038/nrm2774
Hsieh, L. S., Wen, J. H., Miyares, L., Lombroso, P. J., & Bordey, A. (2017). Outbred CD1 mice are as suitable as inbred C57B L/6J mice in performing social tasks. Neurosci Lett, 637, 142-147. https://doi.org/10.1016/j.neulet.2016.11.035
Islam, F., Quadery, T. M., Bai, R., Luckett-Chastain, L. R., Hamel, E., Ihnat, M. A., & Gangjee, A. (2021). Novel pyrazolo[4,3-d]pyrimidine microtubule targeting agents (MTAs): Synthesis, structure-activity relationship, in vitro and in vivo evaluation as antitumor agents. Bioorg Med Chem Lett, 41, 127923. https://doi.org/10.1016/j.bmcl.2021.127923
Janke, C., & Bulinski, J. C. (2011). Post-translational regulation of the microtubule cytoskeleton: mechanisms and functions. Nat Rev Mol Cell Biol, 12(12), 773-786. https://doi.org/10.1038/nrm3227
Janke, C., & Magiera, M. M. (2020). The tubulin code and its role in controlling microtubule properties and functions. Nat Rev Mol Cell Biol, 21(6), 307-326. https://doi.org/10.1038/s41580-020-0214-3
Kapellos, T. S., Taylor, L., Lee, H., Cowley, S. A., James, W. S., Iqbal, A. J., & Greaves, D. R. (2016). A novel real time imaging platform to quantify macrophage phagocytosis. Biochem Pharmacol, 116, 107-119. https://doi.org/10.1016/j.bcp.2016.07.011
Kawahata, I., Finkelstein, D. I., & Fukunaga, K. (2022). Pathogenic Impact of alpha-Synuclein Phosphorylation and Its Kinases in alpha-Synucleinopathies. Int J Mol Sci, 23(11). https://doi.org/10.3390/ijms23116216
Killinger, B. A., Mercado, G., Choi, S., Tittle, T., Chu, Y., Brundin, P., & Kordower, J. H. (2023). Distribution of phosphorylated alpha-synuclein in non-diseased brain implicates olfactory bulb mitral cells in synucleinopathy pathogenesis. NPJ Parkinsons Dis, 9(1), 43. https://doi.org/10.1038/s41531-023-00491-3
Koenigsknecht, J., & Landreth, G. (2004). Microglial phagocytosis of fibrillar beta-amyloid through a beta1 integrin-dependent mechanism. J Neurosci, 24(44), 9838-9846. https://doi.org/10.1523/JNEUROSCI.2557-04.2004
Kuhn, M. (1998). The microtubule depolymerizing drugs nocodazole and colchicine inhibit the uptake of Listeria monocytogenes by P388D1 macrophages. FEMS Microbiol Lett, 160(1), 87-90. https://doi.org/10.1111/j.1574-6968.1998.tb12895.x
L'Hernault, S. W., & Rosenbaum, J. L. (1985). Chlamydomonas alpha-tubulin is posttranslationally modified by acetylation on the epsilon-amino group of a lysine. Biochemistry, 24(2), 473-478. https://doi.org/10.1021/bi00323a034
Lashuel, H. A., Mahul-Mellier, A. L., Novello, S., Hegde, R. N., Jasiqi, Y., Altay, M. F., Donzelli, S., DeGuire, S. M., Burai, R., Magalhaes, P., Chiki, A., Ricci, J., Boussouf, M., Sadek, A., Stoops, E., Iseli, C., & Guex, N. (2022). Revisiting the specificity and ability of phospho-S129 antibodies to capture alpha-synuclein biochemical and pathological diversity. NPJ Parkinsons Dis, 8(1), 136. https://doi.org/10.1038/s41531-022-00388-7
Lee, B. R., & Kamitani, T. (2011). Improved immunodetection of endogenous alpha-synuclein. PLoS One, 6(8), e23939. https://doi.org/10.1371/journal.pone.0023939
Lee, H. J., Khoshaghideh, F., Lee, S., & Lee, S. J. (2006). Impairment of microtubule-dependent trafficking by overexpression of alpha-synuclein. Eur J Neurosci, 24(11), 3153-3162. https://doi.org/10.1111/j.1460-9568.2006.05210.x
Lu, W., & Gelfand, V. I. (2017). Moonlighting Motors: Kinesin, Dynein, and Cell Polarity. Trends Cell Biol, 27(7), 505-514. https://doi.org/10.1016/j.teb.2017.02.005
Mandrekar, S., Jiang, Q., Lee, C. Y., Koenigsknecht-Talboo, J., Holtzman, D. M., & Landreth, G. E. (2009). Microglia mediate the clearance of soluble Abeta through fluid phase macropinocytosis. J Neurosci, 29(13), 4252-4262. https://doi.org/10.1523/JNEUROSCI.5572-08.2009
Manka, S. W., & Moores, C. A. (2018). The role of tubulin-tubulin lattice contacts in the mechanism of microtubule dynamic instability. Nat Struct Mol Biol, 25(7), 607-615. https://doi.org/10.1038/s41594-018-0087-8
Mason, D. M., Nouraei, N., Pant, D. B., Miner, K. M., Hutchison, D. F., Luk, K. C., Stolz, J. F., & Leak, R. K. (2016). Transmission of alpha-synucleinopathy from olfactory structures deep into the temporal lobe. Mol Neurodegener, 11(1), 49. https://doi.org/10.1186/s13024-016-0113-4
Mason, D. M., Wang, Y., Bhatia, T. N., Miner, K. M., Trbojevic, S. A., Stolz, J. F., Luk, K. C., & Leak, R. K. (2019). The center of olfactory bulb-seeded alpha-synucleinopathy is the limbic system and the ensuing pathology is higher in male than in female mice. Brain Pathol, 29(6), 741-770. https://doi.org/10.1111/bpa.12718
Mazzetti, S., Calogero, A. M., Pezzoli, G., & Cappelletti, G. (2023). Cross-talk between alpha-synuclein and the microtubule cytoskeleton in neurodegeneration. Exp Neurol, 359, 114251. https://doi.org/10.1016/j.expneurol.2022.114251
Miner, K. M., Jamenis, A. S., Bhatia, T. N., Clark, R. N., Abbas, M., Luk, K. C., & Leak, R. K. (2023). The variance in phosphorylated, insoluble α-synuclein in humans, rats, and mice is not mainly driven by biological sex. Acta Neuropathol, 146(4), 651-654. https://doi.org/10.1007/s00401-023-02621-w
Miner, K. M., Jamenis, A. S., Bhatia, T. N., Clark, R. N., Rajasundaram, D., Sauvaigo, S., Mason, D. M., Posimo, J. M., Abraham, N., DeMarco, B. A., Hu, X., Stetler, R. A., Chen, J., Sanders, L. H., Luk, K. C., & Leak, R. K. (2022). alpha-synucleinopathy exerts sex-dimorphic effects on the multipurpose DNA repair/redox protein APE1 in mice and humans. Prog Neurobiol, 216, 102307. https://doi.org/10.1016/j.pneurobio.2022.102307
Mitchison, T., & Kirschner, M. (1984). Dynamic instability of microtubule growth. Nature, 312(5991), 237-242. https://doi.org/10.1038/312237α0
Nakayama, K., Suzuki, Y., & Yazawa, I. (2012). Binding of neuronal alpha-synuclein to beta-III tubulin and accumulation in a model of multiple system atrophy. Biochem Biophys Res Commun, 417(4), 1170-1175. https://doi.org/10.1016/j.bbrc.2011.12.092
Naren, P., Samim, K. S., Tryphena, K. P., Vora, L. K., Srivastava, S., Singh, S. B., & Khatri, D. K. (2023). Microtubule acetylation dyshomeostasis in Parkinson's disease. Transl Neurodegener, 12(1), 20. https://doi.org/10.1186/s40035-023-00354-0
Nekooki-Machida, Y., & Hagiwara, H. (2020). Role of tubulin acetylation in cellular functions and diseases. Med Mol Morphol, 53(4), 191-197. https://doi.org/10.1007/s00795-020-00260-8
Niwa, S. (2015). Kinesin superfamily proteins and the regulation of microtubule dynamics in morphogenesis. Anat Sci Int, 90(1), 1-6. https://doi.org/10.1007/si2565-014-0259-5
Nouraei, N., Mason, D. M., Miner, K. M., Carcella, M. A., Bhatia, T. N., Dumm, B. K., Soni, D., Johnson, D. A., Luk, K. C., & Leak, R. K. (2018). Critical appraisal of pathology transmission in the alpha-synuclein fibril model of Lewy body disorders. Exp Neurol, 299(Pt A), 172-196. https://doi.org/10.1016/j.expneurol.2017.10.017
Oikawa, T., Nonaka, T., Terada, M., Tamaoka, A., Hisanaga, S., & Hasegawa, M. (2016). alpha-Synuclein Fibrils Exhibit Gain of Toxic Function, Promoting Tau Aggregation and Inhibiting Microtubule Assembly. J Biol Chem, 291(29), 15046-15056. https://doi.org/10.1074/jbc.M116.736355
Parato, J., & Bartolini, F. (2021). The microtubule cytoskeleton at the synapse. Neurosci Lett, 753, 135850. https://doi.org/10.1016/j.neulet.2021.135850
Park, H., Hong, S., & Hong, S. (2012). Nocodazole is a high-affinity ligand for the cancer-related kinases ABL, c-KIT, BRAF, and MEK. ChemMedChem, 7(1), 53-56. https://doi.org/10.1002/cmdc.201100410
Parra-Rivas, L., A., Madhivanan, K., Wang, L., Boyer, N. P., Prakashchand, D. D., Aulston, B. D., Pizzo, D., P., Branes-Guerrero, K., Tang, Y., Das, U., Scott, D., A, Rangamani, P., & Roy, S. (2022). Serine-129 phosphorylation of α-synuclein is a trigger for physiologic protein-protein interactions and synaptic function. bioRxiv, 2022.2012.2022.521485. https://doi.org/10.1101/2022.12.22.521485
Pellegrini, L., Wetzel, A., Granno, S., Heaton, G., & Harvey, K. (2017). Back to the tubule: microtubule dynamics in Parkinson's disease. Cell Mol Life Sci, 74(3), 409-434. https://doi.org/10.1007/s00018-016-2351-6
Pero, M. E., Chowdhury, F., & Bartolini, F. (2023). Role of tubulin post-translational modifications in peripheral neuropathy. Exp Neurol, 360, 114274. https://doi.org/10.1016/j.expneurol.2022.114274
Polinski, N. K., Volpicelli-Daley, L. A., Sortwell, C. E., Luk, K. C., Cremades, N., Gottler, L. M., Froula, J., Duffy, M. F., Lee, V. M. Y., Martinez, T. N., & Dave, K. D. (2018). Best Practices for Generating and Using Alpha-Synuclein Pre-Formed Fibrils to Model Parkinson's Disease in Rodents. J Parkinsons Dis, 8(2), 303-322. https://doi.org/10.3233/JPD-171248
Portran, D., Schaedel, L., Xu, Z., Thery, M., & Nachury, M. V. (2017). Tubulin acetylation protects long-lived microtubules against mechanical ageing. Nat Cell Biol, 19(4), 391-398. https://doi.org/10.1038/ncb3481
Posimo, J. M., Unnithan, A. S., Gleixner, A. M., Choi, H. J., Jiang, Y., Pulugulla, S. H., & Leak, R. K. (2014). Viability assays for cells in culture. Journal of visualized experiments: JoVE, 83(83), e50645. https://doi.org/10.3791/50645
Prots, I., Veber, V., Brey, S., Campioni, S., Buder, K., Riek, R., Bohm, K. J., & Winner, B. (2013). alpha-Synuclein oligomers impair neuronal microtubule-kinesin interplay. J Biol Chem, 288(30), 21742-21754. https://doi.org/10.1074/jbc.M113.451815
Qureshi, H. Y., & Paudel, H. K. (2011). Parkinsonian neurotoxin 1-methyl-4-phenyl-1,2,3,6-tetrahydropyridine (MPTP) and alpha-synuclein mutations promote Tau protein phosphorylation at Ser262 and destabilize microtubule cytoskeleton in vitro. J Biol Chem, 286(7), 5055-5068. https://doi.org/10.1074/jbc.M110.178905
Ramalingam, N., Jin, S. X., Moors, T. E., Fonseca-Ornelas, L., Shimanaka, K., Lei, S., Cam, H. P., Watson, A. H., Brontesi, L., Ding, L., Hacibaloglu, D. Y., Jiang, H., Choi, S. J., Kanter, E., Liu, L., Bartels, T., Nuber, S., Sulzer, D., Mosharov, E. V., . . . Dettmer, U. (2023). Dynamic physiological alpha-synuclein S129 phosphorylation is driven by neuronal activity. NPJ Parkinsons Dis, 9(1), 4. https://doi.org/10.1038/s41531-023-00444-w
Ren, Y., Liu, W., Jiang, H., Jiang, Q., & Feng, J. (2005). Selective vulnerability of dopaminergic neurons to microtubule depolymerization. J Biol Chem, 280(40), 34105-34112. https://doi.org/10.1074/jbc.M503483200
Rohena, C. C., Risinger, A. L., Devambatla, R. K., Dybdal-Hargreaves, N. F., Kaul, R., Choudhary, S., Gangjee, A., & Mooberry, S. L. (2016). Janus Compounds, 5-Chloro-N(4)-methyl-N(4)-aryl-9H-pyrimido[4,5-b]indole-2,4-diamines, Cause Both Microtubule Depolymerizing and Stabilizing Effects. Molecules, 21(12). https://doi.org/10.3390/molecules21121661
Saez-Calvo, G., Sharma, A., Balaguer, F. A., Barasoain, I., Rodriguez-Salarichs, J., Olieric, N., Munoz-Hernandez, H., Berbis, M. A., Wendeborn, S., Penalva, M. A., Matesanz, R., Canales, A., Prota, A. E., Jimenez-Barbero, J., Andreu, J. M., Lamberth, C., Steinmetz, M. O., & Diaz, J. F. (2017). Triazolopyrimidines Are Microtubule-Stabilizing Agents that Bind the Vinca Inhibitor Site of Tubulin. Cell Chem Biol, 24(6), 737-750 e736. https://doi.org/10.1016/j.chembiol.2017.05.016
Sasaki, A., Arawaka, S., Sato, H., & Kato, T. (2015). Sensitive western blotting for detection of endogenous Ser129-phosphorylated alpha-synuclein in intracellular and extracellular spaces. Sci Rep, 5, 14211. https://doi.org/10.1038/srep14211
Schrödinger Release 2023-1. In. Maestro, Schrödinger, LLC, New York, N Y, 2021.
Scott, D., & Roy, S. (2012). alpha-Synuclein inhibits intersynaptic vesicle mobility and maintains recycling-pool homeostasis. J Neurosci, 32(30), 10129-10135. https://doi.org/10.1523/JNEUROSCI.0535-12.2012
Sharon, R., Goldberg, M. S., Bar-Josef, I., Betensky, R. A., Shen, J., & Selkoe, D. J. (2001). alpha-Synuclein occurs in lipid-rich high molecular weight complexes, binds fatty acids, and shows homology to the fatty acid-binding proteins. Proc Natl Acad Sci USA, 98(16), 9110-9115. https://doi.org/10.1073/pnas.171300598
Toba, S., Jin, M., Yamada, M., Kumamoto, K., Matsumoto, S., Yasunaga, T., Fukunaga, Y., Miyazawa, A., Fujita, S., Itoh, K., Fushiki, S., Kojima, H., Wanibuchi, H., Arai, Y., Nagai, T., & Hirotsune, S. (2017). Alpha-synuclein facilitates to form short unconventional microtubules that have a unique function in the axonal transport. Sci Rep, 7(1), 16386. https://doi.org/10.1038/s41598-017-15575-3
Volpicelli-Daley, L. A. (2017). Effects of alpha-synuclein on axonal transport. Neurobiol Dis, 105, 321-327. https://doi.org/10.1016/j.nbd.2016.12.008
Volpicelli-Daley, L. A., Gamble, K. L., Schultheiss, C. E., Riddle, D. M., West, A. B., & Lee, V. M. (2014). Formation of alpha-synuclein Lewy neurite-like aggregates in axons impedes the transport of distinct endosomes. Mol Biol Cell, 25(25), 4010-4023. https://doi.org/10.1091/mbc.E14-02-0741
Volpicelli-Daley, L. A., Luk, K. C., & Lee, V. M. (2014). Addition of exogenous alpha-synuclein preformed fibrils to primary neuronal cultures to seed recruitment of endogenous alpha-synuclein to Lewy body and Lewy neurite-like aggregates. Nat Protoc, 9(9), 2135-2146. https://doi.org/10.1038/nprot.2014.143
Volpicelli-Daley, L. A., Luk, K. C., Patel, T. P., Tanik, S. A., Riddle, D. M., Stieber, A., Meaney, D. F., Trojanowski, J. Q., & Lee, V. M. (2011). Exogenous alpha-synuclein fibrils induce Lewy body pathology leading to synaptic dysfunction and neuron death. Neuron, 72(1), 57-71. https://doi.org/S0896-6273(11)00844-0 [pii]10.1016/j.neuron.2011.08.033
Warren, J. D., Orr, B., & Compton, D. A. (2020). A comparative analysis of methods to measure kinetochore-microtubule attachment stability. Methods Cell Biol, 158, 91-116. https://doi.org/10.1016/bs.mcb.2020.01.004
Wu, D., Jin, Y., Shapiro, T. M., Hinduja, A., Baas, P. W., & Tom, V. J. (2020). Chronic neuronal activation increases dynamic microtubules to enhance functional axon regeneration after dorsal root crush injury. Nat Commun, 11(1), 6131. https://doi.org/10.1038/s41467-020-19914-3
Xu, Z., Schaedel, L., Portran, D., Aguilar, A., Gaillard, J., Marinkovich, M. P., Thery, M., & Nachury, M. V. (2017). Microtubules acquire resistance from mechanical breakage through intralumenal acetylation. Science, 356(6335), 328-332. https://doi.org/10.1126/science.aai8764
Zhou, C., Yan, L., Zhang, W. H., & Liu, Z. (2019). Structural basis of tubulin detyrosination by VASH2/SVBP heterodimer. Nat Commun, 10(1), 3212. https://doi.org/10.1038/s41467-019-11277-8

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

It is further to be understood that all values are approximate and are provided for description.

All patents, applications, publications, test methods, literature, and other materials cited herein are incorporated by reference. If there is a discrepancy between (a) the incorporated by reference patents, applications, publications, test methods, literature, and other materials, and (b) the present application, then the present application's specification, figures, and claims control the meaning of any terms and the scope of the inventions set forth herein.

What is claimed is:

1. A compound having the structure of either AGN5 or AGN11:

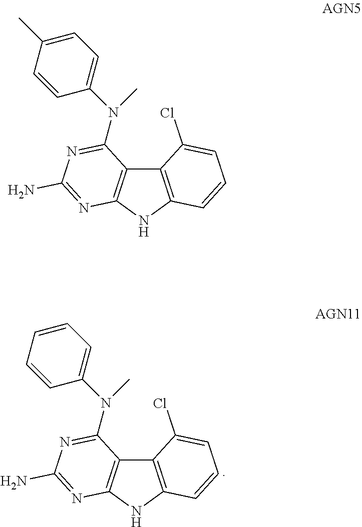 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]

2. A composition comprising a compound having the structure of either AGN5 or AGN11: 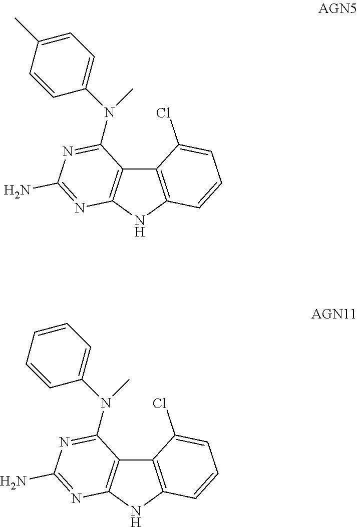 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]
and a pharmaceutically acceptable carrier.

3. A method for treating a patient having a disease selected from the group consisting of dementia with Lewy Bodies, Parkinson's Disease, and Alzheimer Disease with Lewy Bodies comprising administering to said patient a therapeutically effective amount of a compound having a structure of either AGN5 or AGN11: 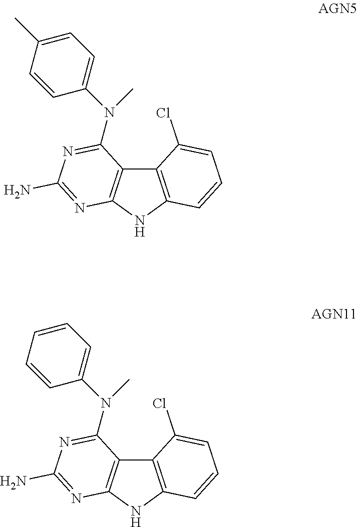 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]
for treating said patient.

4. A method is for treating a patient having a disease selected from the group consisting of dementia with Lewy Bodies, Parkinson's Disease, and Alzheimer Disease with Lewy Bodies comprising administering to said patient a therapeutically effective amount of a composition comprising a compound having a structure of either AGN5 or AGN11: 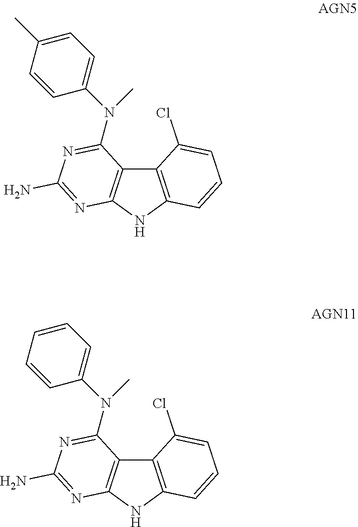 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]
and a pharmaceutically acceptable carrier, for treating said patient.

5. A method for treating a patient affected with a Lewy body disease comprising administering to said patient a therapeutically effective amount of a compound having a structure of either 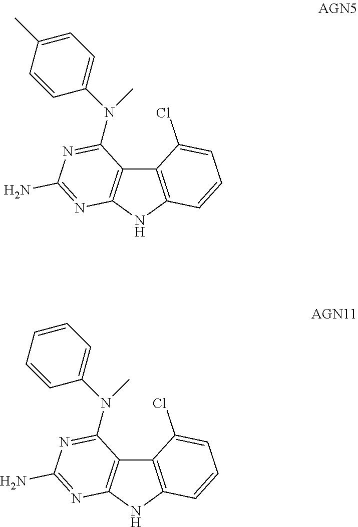 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]
for treating said patient.

6. A method for treating a patient affected with a Lewy body disease comprising administering to said patient a therapeutically effective amount of a composition comprising a compound having a structure of either AGN5 or AGN11: 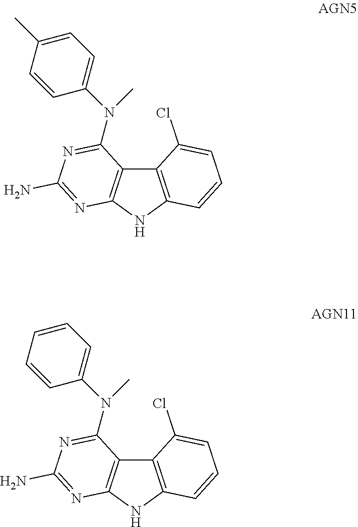 [SMILES: Cc1ccc(N(C)c2nc(N)nc3[nH]c4cccc(Cl)c4c23)cc1 ; CN(c1ccccc1)c1nc(N)nc2[nH]c3cccc(Cl)c3c12]
and a pharmaceutically acceptable carrier, for treating said patient.

7. A compound having the structure of AG161-41, AG161-47, AG182-126, or AG185-174:

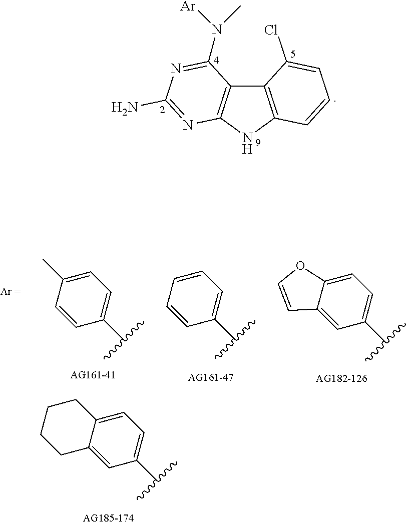

8. A composition comprising a compound having the structure of AG161-41, AG161-47, AG182-126, or AG185-174: 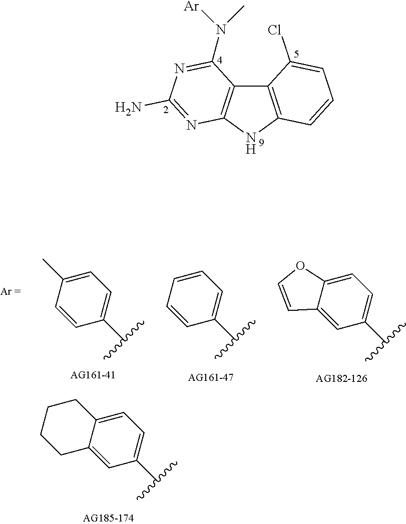
and a pharmaceutically acceptable carrier.

9. A method for treating a patient having a disease selected from the group consisting of dementia with Lewy Bodies, Parkinson's Disease, and Alzheimer Disease with Lewy Bodies comprising administering to said patient a therapeutically effective amount of a compound having a structure of either AG161-41, AG161-47, AG182-126, or AG185-174: 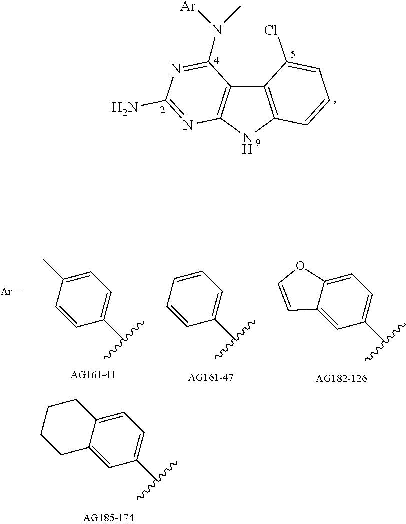
for treating said patient.

10. A method for treating a patient having a disease selected from the group consisting of dementia with Lewy Bodies, Parkinson's Disease, and Alzheimer Disease with Lewy Bodies comprising administering to said patient a therapeutically effective amount of a composition comprising a compound having the structure of AG161-41, AG161-47, AG182-126, or AG185-174: 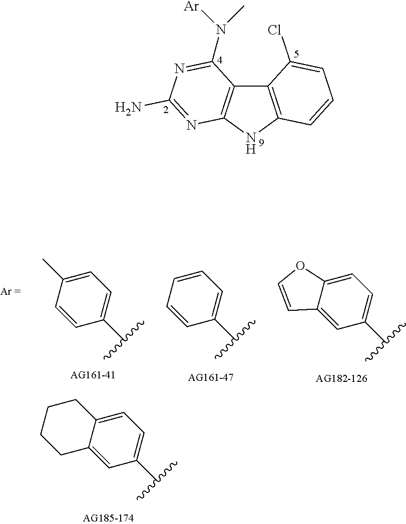
and a pharmaceutically acceptable carrier.
